(19) United States
(12) Patent Application Publication
(10) Pub. No.: US 2024/0368553 A1
(43) Pub. Date: Nov. 7, 2024

(54) PORCINE MUSCLE EXTRACELLULAR MATRIX-DERIVED HYDROGEL AND USES THEREOF

(71) Applicant: University of Connecticut, Farmington, CT (US)

(72) Inventors: Cato T. Laurencin, Farmington, CT (US); Mohammed A. Barajaa, Riyadh (SA)

(21) Appl. No.: 18/652,048

(22) Filed: May 1, 2024

Related U.S. Application Data
(60) Provisional application No. 63/463,096, filed on May 1, 2023.

(51) Int. Cl. C12N 5/077; A61K 35/34; A61P 21/00

(52) U.S. Cl. CPC C12N 5/0658; A61K 35/34; A61P 21/00; C12N 2501/727; C12N 2501/73; C12N 2527/00; C12N 2533/90

(57) ABSTRACT

The present disclosure provides methods for making decellularized skeletal muscle extracellular matrix (smECM) hydrogels, as well as compositions and methods of treatment related to the smECM hydrogels.

### CROSS-REFERENCE TO RELATED APPLICATIONS

This application claims the benefit of U.S. Provisional Patent Application No. 63/463,096, filed May 1, 2023, which is incorporated by reference herein in its entirety.

### STATEMENT REGARDING FEDERALLY SPONSORED RESEARCH

This invention was made with government support under AR079114 awarded by the National Institutes of Health, and 1332329 awarded by the National Science Foundation. The government has certain rights in the invention.

### BACKGROUND

The extracellular matrix (ECM) is the acellular component of any tissue or organ, produced and maintained by resident cells. Numerous cellular functions, such as attachment, proliferation, migration, and differentiation, rely on the ECM. Comprising both structural and nonstructural proteins, the ECM exhibits significant variation across diverse tissues. In recent years, extensive efforts have been made to understanding the distinctive properties of the ECM for each tissue, including structural, mechanical, and compositional characteristics.

This knowledge is important in regenerative engineering, where the objective is often to emulate native ECM compositional, structural, and mechanical properties in a scaffold design to facilitate the regeneration of damaged tissues or organs. Currently, various natural and synthetic scaffold materials have been developed using the biochemical, mechanical, and structural cues from the native ECM as a blueprint. However, despite these advancements, existing natural or synthetic materials remain inadequate in fully replicating the intricate complexity inherent in the natural ECM, and as a result, offer unsatisfactory or suboptimal results.

This is important, for example, for gross muscle damage or loss. While the skeletal muscle exhibits a unique endogenous regenerative capacity upon minor injuries or degradation (<20% muscle loss). However, in cases of severe injuries or degradation (>20% muscle loss), the skeletal muscle's endogenous regenerative capacity is impaired, resulting in irreversible scarring, and permanent functional and structural muscle deficits-a condition known as volumetric muscle loss (VML). There is an unmet need for novel therapies that can, e.g., recover the functional and structural impairments in VML patients and patients otherwise in need of additional functional muscle tissue.

### SUMMARY

The disclosure relates to decellularized, skeletal muscle extracellular matrices and hydrogels made from them, as well as compositions, methods of making, methods of treatment, and uses related thereto. The hydrogels of the disclosure provide a tissue-specific microenvironment suitable for enhanced muscle precursor cell growth and differentiation as compared to known hydrogels.

In one aspect, a method for making a decellularized skeletal muscle extracellular matrix (smECM) is provided, including purifying skeletal muscle tissue to substantially remove non-skeletal muscle tissue comprising blood vessels, fat and facia; comminuting the skeletal muscle tissue into discrete fragments; decellularizing the skeletal muscle tissue by mechanical disruption; digesting nucleic acid present in the skeletal muscle tissue with a DNAse and a RNAse; digesting galactose-alpha-1,3-galactose (α-Gal) antigen present in the skeletal muscle tissue with α-galactosidase; lyophilizing the skeletal muscle tissue; and cryo-milling the lyophilized skeletal muscle tissue to generate decellularized smECM in a powdered form.

In some embodiments, the method further includes digesting the decellularized smECM with a protease; adjusting pH of the decellularized smECM to about 7.4 to make a low temperature, decellularized smECM pre-gel; and incubating the low temperature, decellularized smECM pre-gel at about 37° C. to form a decellularized smECM hydrogel.

In other embodiments, the method further includes digesting the decellularized smECM with a protease; and modifying the decellularized smECM with phenolic functional groups to make a phenolic functionalized, decellularized smECM (smECM-PhF). In some of such embodiments, the method further includes combining horseradish peroxidase with smECM-PhF to make a smECM-PhF pre-gel; and reacting the smECM-PhF pre-gel with hydrogen peroxide to form a smECM-PhF hydrogel.

In some embodiments, no detergent is used for decellularization of the skeletal muscle tissue.

In some embodiments the skeletal muscle tissue is from a non-human mammal, including a pig.

In some embodiments, the mechanical disruption includes at least one freeze-thaw cycle.

In another aspect, a decellularized smECM is provided, produced according to any of the methods for making smECMs as first noted above and described elsewhere herein. In some embodiments, the decellularized smECM is a decellularized smECM-PhF.

In another aspect, a decellularized smECM hydrogel is provided. In some embodiments, the decellularized smECM hydrogel is a decellularized smECM-PhF hydrogel.

In some embodiments, the decellularized smECM hydrogel or decellularized smECM-PhF hydrogel have a total smECM protein content is statistically equivalent to an equivalent smECM that is not decellularized. In some embodiments, sulfated glucosaminoglycan (sGAG) concentration is statistically equivalent to an equivalent smECM that is not decellularized.

In some embodiments of the decellularized smECM hydrogel or decellularized smECM-PhF hydrogel one or more of the parameters (i) time to start gelling, (ii) time to 50% gelation, or (iii) time to 95% gelation is statistically equivalent to time to the start gelling, time to 50% gelation, or time to 95% gelation, respectively, for an equivalent smECM that is not decellularized.

In another aspect, compositions including at least one of decellularized smECM hydrogel and decellularized smECM-PhF hydrogel are provided.

In another aspect, a method for treating volumetric muscle loss (VML) is provided, including injecting in a subject with VML a therapeutically effective amount of a composition that includes at least one of decellularized smECM hydrogel and decellularized smECM-PhF hydrogel at at least one site of VML in the subject.

In some embodiments, the method instead includes implanting in a subject with VML a therapeutically effective amount of a composition that includes at least one of decellularized smECM hydrogel and decellularized smECM-PhF hydrogel at at least one site of VML in the subject. In some embodiments, the implanted composition is pre-formed in a shape complementary to muscle absent due to VML, and in some embodiments, the implanted composition is of a standardized shape.

These and other aspects of the present invention are described in more detail below.

### BRIEF DESCRIPTION OF THE DRAWINGS

The accompanying drawings, which are included to provide a further understanding of the disclosure, are incorporated in and constitute a part of this specification, illustrate embodiments of the disclosure and together with the detailed description serve to explain the principles of the invention. No attempt is made to show structural details of the invention in more detail than may be necessary for a fundamental understanding of the invention and various ways in which it may be practiced.

FIG. 1 shows a schematic illustration of the various steps involved in the decellularization of the skeletal muscle tissue for the production of skeletal muscle extracellular matrix (smECM)-derived hydrogels. (A) Porcine hindlimb muscles are harvested, and (B) purified by removal of the blood vessels, fat, and facial tissues. (C) The purified muscle tissues are then grounded into small fragments, and (D) stirred in DI water to remove excess blood from the tissue. (E) The muscle fragments are then decellularized by mechanical disruption (MD), or by treatment with Triton X-100, or sodium deoxycholate (SDC) (two previously used decellularization methods). (F) Next, the decellularized muscle tissues are treated with isopropanol to dissolve any remnant fat in the sample, followed by (G) treatment with DNAse and RNAse to degrade any remnant DNA in the sample. (H) Next, the samples are treated with α-Galactosidase to cleave the α-gal antigen in the decellularized tissue, followed by (I) extensively washing the tissues in DI water to remove any detergents/enzyme residues. (J) Post decellularization, the samples are aliquoted and stored at −80 C for later use.

FIG. 2 shows the gross appearance and the various steps involved in the preparation of the different smECM hydrogels. To prepare smECM hydrogels, (column A) the decellularized smECMs were (column B) lyophilized, then (column C) cryo-milled into fine powder. Next, the cryo-milled smECMs were (column D) solubilized in an acidic-based pepsin solution and (column E), and PH neutralized to make smECM pre-gels. The smECM pre-gels maintained a solution state at temperatures below 15° C. and transformed into (column F) a gel state after incubation at 37° C. for 15-30 min. Regardless of the decellularization protocol followed, all pre-gels were able to form hydrogels. All hydrogels were prepared at a similar final concentration of 4 mg/mL. The native group did not undergo any decellularization and was added as a positive control group.

FIG. 3A and FIG. 3B show DNA qualitative and quantitative assessments. (FIG. 3A) The efficacy of the different decellularization protocols in removing the DNA was qualitatively assessed via H&E and DAPI staining to detect the presence of any nuclei in the formed hydrogels. Images are presented at low (10×) and high (20×) magnifications. (FIG. 3B) PicoGreen was used to quantify the total DNA content in the finally-formed hydrogels. Scale bars (100 μm).

FIG. 4A to FIG. 4C show α-Gal epitope assessment. M86 antibody-stained sections from the (FIG. 4A) native non-decellularized hydrogel group and (FIG. 4B) MD, Triton X-100, and SDC hydrogel groups before and (FIG. 4C) after treatment with the enzyme α-Galactosidase. Scale bar (100 μm).

FIG. 5 shows a heat map illustrating the growth factors content in the different smECM hydrogels as assessed via the Quantibody® Growth Factor Array Kit. Each growth factor is highlighted with a rectangular box colored with its corresponding function.

FIG. 6A to FIG. 6C show Quantitative biochemical evaluations of the different smECM hydrogels. (FIG. 6A) total protein, (FIG. 6B) sGAG, and (FIG. 6C) collagen contents.

FIG. 7A to FIG. 7C show Qualitative biochemical evaluations of the different smECM hydrogels via histological staining. (FIG. 7A) Alcan blue and (FIG. 7B) picrosirius red-stained sections were captured at low (10×) and high (20×) magnifications. (FIG. 7C) Collagen nativity assessment. Picrosirius Red-stained sections were imaged under a polarized lens to distinguish the collagen phenotype in the formed smECM hydrogels from the different groups. Images are presented at low (10×) and high (20×) magnifications. Scale bars (100 μm).

FIG. 8A and FIG. 8B show qualitative biochemical evaluations of the different smECM hydrogels via immunohistochemistry staining. (FIG. 8A) laminin and (FIG. 8B) fibronectin-stained sections captured at low (10×) and high (20×) magnifications. Scale bars (100 μm).

FIG. 9A to FIG. 9F show the impact of the decellularization protocol followed on the gelation kinetics and mechanical properties of the produced smECM hydrogels. (FIG. 9A to FIG. 9F) Gelation kinetics evaluations of the different smECM hydrogels via turbidimetric analysis: (FIG. 9A) raw data, (FIG. 9B) normalized data. Normalized data were used to calculate (FIG. 9C) tlag (Time elapsed before the hydrogel starts gelling), (FIG. 9D) t1/2 (Time elapsed before the hydrogel reaches 50% gelation), (FIG. 9E) 195 (Time elapsed before the hydrogel reaches 95% gelation), and (FIG. 9F) S (Gelation velocity). Data show that MD hydrogels showed significantly better gelation kinetics compared to hydrogels produced via Triton X-100 and SDC treatments.

FIG. 10A to FIG. 10C show gross morphology and ultra-structure of the different smECM hydrogels. (FIG. 10A) The gross morphology and (FIG. 10B) ultra-structure of the different smECM hydrogels under SEM captured at low (1000×) and high (3000×) magnifications. (FIG. 10C) SEM images were used to quantify the fibril density in the different smECM hydrogels.

FIG. 11A and FIG. 11B show migration of satellite stem cells (SSCs) toward the different chemoattractants. (FIG. 11A) A schematic illustrating the trans-well-based system used to assess the migration of SSCs toward the different chemoattractants. (FIG. 11B) The degree of SSCs migration toward the different chemoattractants.

FIG. 12A and FIG. 12B show the cyto-compatibility of the different smECM hydrogels. (FIG. 12A) Confocal images of SSCs embedded within the different smECM hydrogels and labeled with Calcein AM (green) for live cells and Ethidium Homodimer-1 (red) for dead cells after days 3 and 7 of culture. (FIG. 12B) Growth of SSCs within the different smECM hydrogels at days 3 and 7 of culture as measured by MTS (@P<0.0001 and % P<0.001 compared to day 3). Scale bar (100 μm).

FIG. 13A and FIG. 13B show overtime monitoring of cell-mediated contraction of the different smECM hydrogels (free-floating hydrogels). (FIG. 13A) Photographic images showing cell-mediated contraction over time and (FIG. 13B) quantification of change in hydrogel area over time.

FIG. 14 shows myo-inductivity of the different smECM hydrogels. Immunofluorescence staining images for MHC showing the myogenic differentiation of SSCs in the different smECM hydrogels. Scale bar (50 μm).

FIG. 15A to FIG. 15D show the overall design of the biocompatibility and immunogenicity in vivo assessment of the smECM hydrogel in the immunocompetent xenotransplantation model. (FIG. 15A) An insulin syringe was used to inject the smECM hydrogel. (FIG. 15B) Each mouse was injected with only one type of biomaterial and received four 350 μL evenly spaced subcutaneous injections in the dorsal region. (FIG. 15C) The overall timeline of the in vivo study which aimed to evaluate the innate (Day 3), mid-phase (Day 7), and late-phase (Day 14) immune responses post-biomaterial injection. (FIG. 15D) At the aforementioned time points, two of the four injections were harvested for histological and IHC analyses, and the remaining two were harvested for flow cytometry analysis.

FIG. 16A and FIG. 16B show cellular infiltration assessment. (FIG. 16A) H&E images showing the local tissue immune response at days 3, 7, and 14 post-subcutaneous injections of the different hydrogels in the xenotransplantation animal model. (FIG. 16B) Quantification of the total number of infiltrated cells. The dotted black lines at the middle of each image separate the subcutaneous tissue (referred to as “ST”) from the injected hydrogels (referred to as “H”). The dotted blue squares inserted on the H&E images of the native group highlight the presence of foreign body giant immune cells (FBGICs). The solid blue squares are the higher magnification of the dotted blue squares. Scale bar for the H&E images (100 μm), whereas the scale bar for the magnified H&E images (50 μm).

FIG. 17A to FIG. 17C show polarized macrophage infiltration. (FIG. 17A) Representative IHC images for CD86 (M1 macrophage marker) and CD163 (M2 macrophage marker) showing macrophage infiltration and the phenotype of the infiltrated macrophages in the different hydrogels at days 3, 7, and 14 post-subcutaneous injections. DAPI was used to label the cellular nuclei (blue). Flow cytometry was used to quantify the percentages of the infiltrated M1 and M2 macrophages, and the acquired percentages were used to calculate the ratio of M2/M1. M2/M1 ratio calculations using (FIG. 17B) CD163 and CD86, and (FIG. 17C) Arginase (M2 macrophage marker) and iNOS (M1 macrophage marker). Scale bar (50 μm).

FIG. 18A to FIG. 18C shows T-helper and T-Cytotoxic T-cell infiltration. (FIG. 18A) Representative IHC images for CD4 (T-helper T-cell marker) and CD8 (T-Cytotoxic T-cell marker) showing T-cell infiltration and the phenotype of the infiltrated T-cells in the different hydrogels at days 3, 7, and 14 post-subcutaneous injections. DAPI was used to label the cellular nuclei (blue). (FIG. 18B) Ratio calculations of T-help/T-Cyto using the percentages of CD4+ (T-helper) and CD8+ (T-Cytotoxic) T-cells as measured by flow cytometry. (FIG. 18C) Ratio calculations of Th2/Th1 using the percentages of IL-4Rα+ (Th2) and IFN-γ+ (Th1) T-cells as measured by flow cytometry. Scale bar (50 μm).

### DETAILED DESCRIPTION

The regeneration of large muscle defects remains a significant challenge in medicine. To date, there is no treatment option for muscle injuries superior to biological grafts, such as autografts and allografts. However, such grafts have several drawbacks that make them risky or unsuitable for long-term use. Autografts, which are sourced from the patient in need of treatment, can cause pain and related complications in patients, assuming adequate muscle stores are available to harvest. Allografts pose the risk of disease transmission and immune rejection. Recently, hydrogels have been developed as potential candidates for muscle tissue regeneration applications, including collagen, fibrin, agarose, silk, alginate, and GelMA-based hydrogels. While such hydrogels have shown regenerative potential, they also have drawbacks, including a lack the structural, biochemical, and biological complexity of natural ECMs, resulting in cells often failing to perform well within such hydrogels. Extracellular matrix-derived hydrogels address some of these shortcomings at the expense of inheriting others, including a lack of tissue-specificity, potential for immune rejection, or, when stripped of antigenic determinants, an incomplete or inadequate composition of ECM proteins due to the preparation of the ECM hydrogel, and the like.

Decellularized, skeletal muscle extracellular matrices and hydrogels made from them are provided herein, as well as related compositions, methods of making, methods of treatment, and uses. The hydrogels of the disclosure provide a tissue-specific microenvironment suitable for enhanced muscle precursor cell growth and differentiation as compared to known hydrogels.

A number of terms are introduced below:

The term “extracellular matrix” and “ECM” refer to a natural scaffolding for cell growth. Natural ECMs (ECMs found in multicellular organisms, such as mammals and humans) are complex mixtures of structural and non-structural biomolecules, including, but not limited to, collagens, clastins, laminins, glycosaminoglycans, proteoglycans, antimicrobials, chemoattractants, cytokines, and growth factors. In mammals, ECM often comprises about 90% collagen, in its various forms. The composition and structure of ECMs vary depending on the source of the tissue.

The term “decellularized” means that cells and cell components such as membrane and DNA material and other cell components have been removed from the muscle tissue. Furthermore, as used herein, the “decellularized” when used alone includes fully and partially decellularized, where “fully decellularized” muscle tissue contains less than about 50 nanograms (ng) of nuclear (DNA) material per milligram (mg) decellularized muscle tissue, and “partially decellularized” muscle tissue contains less than 50% by weight of the total endogenous nuclear (DNA) material originally present in the unprocessed source muscle tissue.

A “decellularized smECM” refers to a skeletal muscle-derived ECM wherein the endogenous cells have been substantially removed. In various embodiments, the decellularized ECM is isolated or separated from at least about 60%, 70%, 80%, 90%, 95%, 99%, or more, of endogenous cellular material. The presence or extent of endogenous cellular material can be determined using any method known in the art, e.g., Western blotting, detection of nuclei, microscopy, etc.

To “remove” antigens from a smECM, or for a smECM to be “free” or “substantially free” of antigens refers to a smECM in which the endogenous antigen components (e.g., proteins, lipids, carbohydrates (e.g., galactose-alpha-1,3-galactose (α-Gal)), nucleic acids) have been substantially removed. In various embodiments, the decellularized smECM is isolated or separated from at least about 60%, 70%, 80%, 90%, 95%, 99%, or more, of endogenous antigen components. The presence or extent of endogenous antigen components can be determined using any method known in the art, e.g., immunoassays, Western blotting, ELISA, gel electrophoresis, lymphocyte proliferation assays, etc. In various embodiments, ECMs that are substantially free of endogenous antigens do not elicit a significant or destructive immune response, e.g., a xenogeneic immune response, in vitro or in vivo, directed against the smECM.

The terms “delipidized” and “delipidizing” are used herein to mean the characteristic and process by which at least a portion of the lipids naturally present in a tissue are removed from the tissue.

The terms “myofiber” and “myocyte” are used interchangeably and refer to the large, highly specialized cells which are the basic cellular unit of skeletal muscle tissue, which also contains extracellular matrix material and other components. Myofibers often have multiple nucleii and range in size from less than 100 to a few hundred microns (μm) in diameter and have lengths of from a few millimeters to a few centimeters. Myofibers are bundled together into “fascicles,” which are typically sheathed in connective tissue. “Myofibrils” are different from myofibers. Myofibrils are basic rod-like units of myofibers and myofibers typically contain many chains of myofibrils. Decellularization is performed to remove myofibers and other cells, as well as components of myofibers and other cells (e.g., DNA, cytoplasm, membrane material, etc.) from muscle tissue.

As used herein, the term “myoblast” means an embryonic cell that becomes a cell of fiber, or myocyte.

As used herein, the terms “myogenic” and “myogenesis” mean the ability (potential) and process, respectively, of forming new muscle tissue, such as by myoblasts or other muscle-forming cell lineages.

As used herein, the terms “myoinductive” and “myoinduction” mean the ability (potential) and process, respectively, by which primitive, undifferentiated and pluripotent cells are stimulated, such as by soluble factors, extracellular matrix, etc., to develop into a muscle-forming cell lineage. For example, a myoinductive factor is one that stimulates primitive, undifferentiated and pluripotent cells to develop into a muscle-forming cell lineage.

The terms “treatment” or “treating” and their grammatical equivalents refer to the medical management of a subject with an intent to cure, reverse, ameliorate, stabilize, or prevent a disease, condition, or disorder. Treatment may include active treatment, that is, treatment directed specifically toward the improvement of a disease, condition, or disorder. Treatment may include causal treatment, that is, treatment directed toward removal of the cause of the associated disease, condition, or disorder. In addition, this treatment may include palliative treatment, that is, treatment designed for the relief of symptoms rather than the curing of the disease, condition, or disorder. Treatment may include preventative treatment, that is, treatment directed to minimizing or partially or completely inhibiting the development of a disease, condition, or disorder. Treatment may include supportive treatment, that is, treatment employed to supplement another specific therapy directed toward the improvement of the disease, condition, or disorder. In some embodiments, a condition may be pathological. In some embodiments, a treatment may not completely cure.

When introducing elements of the present invention or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. The term “exemplary” is not intended to be construed as a superlative example but merely one of many possible examples.

Recitation of ranges of values herein are merely intended to serve as a shorthand method of referring individually to each separate value falling within the range, unless otherwise indicated herein, and each separate value is incorporated into the specification as if it were individually recited. For example, if a size range is stated as 1 nm to 100 nm (or concentrations, degrees, mass amounts, and the like), it is intended that values such as 2 nm to 90 nm, 10 nm to 70 nm, 30 nm to 95 nm, 75 nm to 100 nm, or 2 nm to 27 nm, etc., are expressly enumerated in this specification. These are only examples of what is specifically intended, and all possible combinations of numerical values between and including the lowest value and the highest value enumerated are to be considered to be expressly stated in this disclosure.

Furthermore, when “about”, “approximately” and/or “substantially” is/are utilized to describe a value, this is meant to encompass minor variations (up to +/−10%) from the stated value. Where no stated value is provided, an element described as “substantially” means at least about 60%, 70%, 80%, 90%, 95%, 99%, or more of the element, as is logically coherent within in the context. Unless specifically stated to the contrary, for ranges specified using “about” language, the about applies to both ends of the recited range whether specified or not. For example, “between about 10 mM and 10 μM” is equivalent to “between about 10 mM and about 10 μM”.

In general, methods of preparing an smECM-derived hydrogel require the isolation of smECM from an animal of interest and from, in the case of an smECM, skeletal muscle. In some embodiments, the skeletal muscle is from mammal. In some embodiments, the smECM is isolated from a vertebrate animal, for example, and without limitation, human, pig, monkey, cattle, and sheep.

The methods described herein involve preparation of a decellularized smECM matrix, as well as for converting the smECM matrix into a hydrogel. The smECM-derived hydrogel is reverse gelling, or can be said to exhibit reverse thermal gelation, in that it forms a gel (sol to gel transition) upon an increase in temperature. The lower critical solution temperature (LCST) in a reverse gel is a temperature below which a reverse-gelling polymer is soluble in its solvent (c.g. water or an aqueous solvent). As the temperature rises above the LOST in a reverse gel, a hydrogel is formed. The general concept of reverse gelation of polymers and its relation to LCST are broadly known in the chemical arts. The smECM hydrogels described herein are prepared, for example from decellularized smECM, as described in more detail below, by digestion of the smECM material with an acid protease, neutralization of the material to form a pre-gel, and then raising the temperature of the pre-gel above the LCST of the pre-gel to cause the pre-gel to hydrogel. The transition temperature for acid-protease-digested from solution to gel is typically within the range of from about 10° C. to about 40° C., and any increments or ranges therebetween, for example from 20° C. to 35° C. For example, the pre-gel can be warmed to about 37° C. to form a hydrogel.

In one aspect, a method for making a decellularized smECM is provided, including purifying skeletal muscle tissue to substantially remove non-skeletal muscle tissue such as blood vessels, fat and facia; comminuting the skeletal muscle tissue into discrete fragments; decellularizing the skeletal muscle tissue by mechanical disruption; digesting nucleic acid present in the skeletal muscle tissue with a DNAse and a RNAse; digesting galactose-alpha-1,3-galactose (α-Gal) antigen present in the skeletal muscle tissue with α-galactosidase; lyophililzing the skeletal muscle tissue; and comminuting the lyophilized skeletal muscle tissue to generate decellularized smECM in a powdered form.

Blood vessels, fat, and facia may be removed by methods known in the art. For example, certain blood vessels and facia and fat can be removed from skeletal muscle tissue, e.g., manually, mechanically, by chemical extraction, and the like.

As used herein, the term “comminute” and any other word forms or cognates thereof, such as, without limitation, “comminution” and “comminuting”, refers to the process of reducing larger particles into smaller particles, including, without limitation, by grinding, blending, shredding, slicing, milling, cutting, shredding. ECM can be comminuted while in any form, including, but not limited to, hydrated forms, frozen, air-dried, lyophilized, powdered, sheet-form. In some embodiments, comminuting the skeletal muscle means grinding one or more times in a meat grinder. In some embodiments, comminuting the lyophilized, decellularized skeletal muscle tissue indicates milling, which in some embodiments is cryo-milling.

Decellularizing the smECM by mechanical disruption can include physical treatments that lyse, kill, and remove cells from an ECM or portion thereof. In some embodiments, mechanical disruption may utilize temperature, force, pressure, and/or electrical disruption. In some embodiments, temperature-based methods may be used in a rapid freeze-thaw mechanism. In some embodiments, for example, by freezing a tissue, microscopic ice crystals may form around a plasma membrane and a cell may be lysed. In some embodiments, after lysing one or more cells, a tissue may be further exposed to liquidized chemicals that may degrade and wash out any residual or undesirable components. In some embodiments, temperature-based methods may conserve a physical structure or characteristic of an smECM. In some embodiments, a skeletal muscle tissue may be decellularized at a suitable freeze temperature. In some embodiments, a suitable freezing temperature may be from about −180° C., −170° C., −160° C., −150° C., −140° C., −130° C., −120° C., −100° C., −90° C., −80° C., −70° C., −60° C., −50° C., −40° C., −30° C., or-20° C., for a length of time including about 0.5 hours, about 1.0 hours, about 1.5 hours, about 2.0 hours, about 2.5 hours, about 3.0 hours, about 4.5 hours, about 5.0, about 5.5 hours, about 6.0 hours, about 6.5 hours, about 7.0 hours, about 7.5 hours, about 8.0 hours, about 8.5 hours, about 9.0 hours, about 9.5 hours, about 10 hours, about 10.5 hours, about 11.0 hours, about 11.5 hours, about 12 hours, about 15 hours, about 18.0 hours, about 24, or about 48 hours, or indefinitely. In embodiments, after a freezing cycle, the sample is then warmed and, optionally, dried, until the sample is substantially or completely thawed. In embodiments, the thawing process is carried out slowly (e.g., while on ice or at a temperature between freezing and room temperature), or more quickly, e.g., at a temperature elevated above room temperature. Such a freeze-thaw process can be carried out 1, 2, 3, 4, 5, 6, 7, 8, 9, 10 or more times. In some embodiments, buffer is changed after one or more thaws. In some embodiment, the mechanical disruption includes freezing at

In some embodiments, a physical treatment may also include a use of pressure. In some embodiments, pressure decellularization may involve a controlled use of hydrostatic pressure applied to skeletal muscle tissue. In some embodiments, pressure decellularization may be performed at high temperatures to avoid unmonitored ice crystal formation. In some embodiments, electrical disruption of a tissue may be performed. In some embodiments, electrical disruption may be done to lyse cells housed in skeletal muscle tissue. In some embodiments, by exposing a tissue to electrical pulses, micropores may be formed at a plasma membrane. In some embodiments, one or more cells may die after their homeostatic electrical balance can be ruined through such applied stimulus. In some embodiments, mechanical disruption may include sonication to enhance decellularization.

In some embodiments, muscle fragments were placed in a vessel with sterile DH₂O and 1% Penicillin/Streptomycin and subjected to 7 freeze-thaw cycles at −80° C. (12 hours freezing, followed by thawing at room temperature until completely thawed then stirring for 1 hour at RT). In some embodiments, the DH₂O solution was replaced with a fresh one at each frecze-thaw cycle.

In some embodiments, for example, after freeze/thawing lyses a cellular membrane, endonucleases and/or exonucleases may degrade nucleic acid content, while other components of a cell may be solubilized and washed away.

In some embodiments, decellularization may include treatment with one or more enzymes such as, without limitation, one or more nucleases (DNAse, RNAse), collagenases, one or more dispases, one or more proteases, and any combination thereof. In some embodiments, the skeletal muscle tissue is treated with DNAse and RNAse.

As α-Gal antigen (α1,3 Gal epitopes) is the primary xeno-antigen that causes hyperacute rejection of non-human animal ECM-derived materials in humans, and even remnant α-Gal antigen in animal ECM-derived materials elicits adverse immunological responses in the human body, in embodiments the skeletal muscle tissue is treated with α-galactosidase, which specifically cleaves the α-Gal antigen from tissues without masking their regenerative potential. In some embodiments, between 1 U/l and 100 U/l, 1 U/l and 75 U/1, 1 U/l and 50 U/1, 5 U/l and 50 U/l, 10 U/l and 40 U/l, 10 U/l and 30 U/l α-galactosidase is used, and in some embodiments, 20 U/l is used to digest the α-Gal antigen.

The supernatant is then concentrated by drying, for example by spraying or lyophilization, and then can be stored long term, reconstituted in a solution, e.g., an aqueous solution, such as water, saline, isotonic buffer, PBS, and the like. Lyophilization may occur at room temperature or at below room temperature, for example at 0° C., −10° C., −20° C., −30° C., and lower.

In some embodiments, the lyophilized skeletal muscle tissue is further processed by comminution, e.g. milling or cryo-milling.

In some embodiments, the method further includes digesting the decellularized smECM with a protease. Suitable proteases include acid proteases, such as pepsin, trypsin or combinations thereof. Reactions with such acid proteases are carried out at a low pH, e.g., between about pH 1 and pH 6, or pH 1 and pH4.

To produce a gel form of the smECM for cell culture or in vivo therapy, the temperature of the solution is raised to a desired temperature and the pH of the solution comprising the smECM is raised to a pH between 7.2 and 7.8. The pH can be raised by adding one or more of a base or an isotonic buffered solution, for example and without limitation, NaOH or PBS at pH 7.4.

As used herein, the term “isotonic buffered solution” refers to a solution that is buffered to a pH between 7.2 and 7.8, e.g., pH 7.4, and that has a balanced concentration of salts to promote an isotonic environment. As used herein, the term “base” refers to any compound or a solution of a compound with a pH greater than 7. For example and without limitation, the base is an alkaline hydroxide or an aqueous solution of an alkaline hydroxide. In certain embodiments, the base is NaOH or NaOH in PBS.

In some embodiments, the decellularized smECM concentration can be about 1-20 mg/mL, or about 2-8 mg/mL, or, in some embodiments, about 4 mg/mL. The solution comprising the skeletal muscle extracellular matrix can then be injected through a high gauge needle, into injured tissue or any tissue in need. At body temperature (about 37° C.), such solution then forms into a gel. Cells, drugs, proteins, or other therapeutic agents can also be delivered inside the decellularized smECM gel.

Alternatively, in some embodiments, the decellularized smECM can be induced to form a gel in a mold of a desired size and shape to produce an implant that can be implanted into a patient in need.

In other embodiments, the method further includes digesting the decellularized smECM with a protease; and modifying the decellularized smECM with phenolic functional groups to make a phenolic functionalized, decellularized smECM (smECM-PhF). In some of such embodiments, the method further includes combining horseradish peroxidase with smECM-PhF to make a smECM-PhF pre-gel; and reacting the smECM-PhF pre-gel with hydrogen peroxide to form a smECM-PhF hydrogel.

In various embodiments, the use of detergents is avoided for the decellularization process, or from all processing of the decellularized smECM, the decellularized smECM pre-gel or the decellularized smECM hydrogel.

In another aspect, a decellularized smECM, a decellularized smECM pre-gel, and a decellularized smECM hydrogel is provided, produced according to any of the methods for making smECMs described herein. In some embodiments, the decellularized smECM (as well as pre-gel and hydrogel), includes a crosslinkable phenolic derivative of smECM, smECM-PhF.

Advantages of the decellularized smECM methods herein include a more native-like ECM composition, where more of the ECM protein content and protein composition is maintained compared to other preparation methods, e.g., those that rely on the use of detergents in the decellularization process. Thus, in some embodiments, the decellularized smECM hydrogel or decellularized smECM-PhF hydrogel have a total smECM protein content that is statistically equivalent to an equivalent smECM that is not decellularized, i.e., a native ECM. In some embodiments, the sulfated glucosaminoglycan (sGAG) concentration is statistically equivalent to an equivalent smECM that is not decellularized.

Another advantage of the methods described herein and resulting decellularized smECM hydrogels is more native-like gelling behavior. In some embodiments, one or more of the parameters (i) time to start gelling, (ii) time to 50% gelation, or (iii) time to 95% gelation is statistically equivalent to time to the start gelling, time to 50% gelation, or time to 95% gelation, respectively, for an equivalent smECM that is not decellularized.

The term “statistically equivalent,” as used here, means any observed difference between a decellularized smECM as described here and a native smECM from the same animal tissue is of a p value of >0.05 when a one-way analysis of variance (one-way ANOVA) and/or two-way analysis of variance (two-way ANOVA) with Tukey's post hoc test is performed.

In another aspect, the disclosure provides compositions and methods including injecting or implanting in a subject in need thereof an effective amount of a composition that includes decellularized smECM hydrogel and/or decellularized smECM-PhF hydrogel. In some embodiments, the disclosure provides a method that includes injecting or implanting in a subject in need thereof a composition that includes decellularized smECM hydrogel and/or decellularized smECM-PhF hydrogel. In certain embodiments, the injection or implantation of the composition repairs damage to skeletal muscle tissue sustained by said subject. In other embodiments, the injection or implantation of said composition repairs damage caused by ischemia in the subject. In some embodiments, the composition comprising the decellularized smECM or decellularized smECM-PhF material can degrade within about one month, two months, or three months following injection or implantation. In certain embodiments, the injection or implantation of the composition repairs damage to skeletal muscle tissue sustained by the subject. In certain embodiments, the injection or implantation of the composition repairs damage caused by ischemia in said subject. Herein, an effective amount can be an amount that increases blood flow in the area of the injection or implantation of the treated subject. In some instances an effective amount is an amount that increases blood flow as measured by, e.g., Doppler waveform analysis, pulse volume recording, duplex arterial ultrasound study, or exercise Doppler stress testing. In some instances, an effective amount is an amount that increases muscle mass in the area of the injection or implantation of the treated subject. In some instances, an effective amount is an amount that induces new vascular formation in the area of the injection or implantation of the treated subject.

In some embodiments, the composition can include one or more biocompatible carriers. As is understood by persons of ordinary skill in the relevant art, a carrier may be biologically inert or inactive. A carrier may be biologically active, for example, in a manner which enhances the muscle-forming potential of the decellularized smECM, or the carrier may provide or induce another biological activity, property or effect which may be unrelated, complementary or supplemental to the muscle-forming potential of the decellularized smECM.

Suitable biocompatible carriers may be naturally occurring or derived therefrom, or synthetic, or a combination of such materials. Generally, suitable biocompatible carriers include for example, without limitation, buffered solutions, glycerol, hyaluronate, polyethylene glycol, stearates, cellulose-derived materials (e.g., chitosan, alginates, hydroxypropyl cellulose (HPC), carboxymethyl cellulose (CMC), hydroxypropyl methyl cellulose (HPMC), etc.), and combinations thereof. Particularly suitable biocompatible carriers for use with the decellularized muscle matrices include, without limitation, hyaluronate, glycerol and buffered solutions. In some embodiments, the biocompatible carrier comprises sodium hyaluronate. In some embodiments, the biocompatible carrier comprises sodium hyaluronate and a buffered saline solution. In some embodiments, the biocompatible carrier comprises glycerol.

In some embodiments, the carrier includes at least one of an isotonic solution, a sodium chloride solution, lactated Ringer's solution, a phosphate-buffered saline solution (PBS), platelet rich plasma (PRP), bone marrow aspirate (BMA), and hyaluronic acid (HA) or a derivative thereof such as sodium hyaluronate. In embodiments, the carrier is a sodium chloride solution at a concentration of about 0.1% to about 1%.

In some embodiments, the carrier comprises thrombin. In some embodiments, the carrier comprises fibrin. In some embodiments, the carrier comprises glycerin. In some embodiments, the carrier comprises gelatin. In some embodiments, the carrier comprises collagen. In some embodiments, the carrier comprises lecithin. In some embodiments, the carrier comprises a sugar. In some embodiments, the sugar comprises a polysaccharide. In some embodiments the carrier includes a combination of two or more carrier components.

The compositions herein can include a decellularized smECM and another component or components. In some instances, the amount of smECM in the total composition is greater than 90% or 95% or 99% of the composition by weight. In some embodiments, the smECM in the total composition is greater than 1%, 5%, 10%, 20%, 30%, 40%, 50%, 60%, 70%, or 80% of the composition by weight.

In some embodiments, the composition can be injectable. The composition can be formulated into a powder or particulate. In other instances, the composition can be formulated to be in liquid form at room temperature, typically 20° C. to 25° C., and in gel form at a temperature greater than room temperature or greater than about 35° C. In some instances, the composition is configured to be delivered to a tissue parenterally, such as through a small gauge needle (e.g., 27 gauge or smaller). In some instances, said composition is suitable for direct implantation into a patient. The composition can be formulated either in a dry or hydrated form to be placed on or in wounds. Dosage amounts and frequency can routinely be determined based on the varying condition of the injured tissue and patient profile. At body temperature, the solution can then form into a hydrogel.

In some embodiments, the decellularized smECM pre-gel can be placed into a mold of a predetermined initial shape or form and converted into a hydrogel of that shape or form. For example, in some embodiments, the decellularized smECM pre-gel can be placed in a mold having a desired predetermined three-dimensional shape (e.g., shect, brick, sphere, cylinder, disk, conical sections, asymmetric, irregular, etc.) and at least partially gelled by, e.g., raising the temperature to or above the LCST to produce a composition having that predetermined three-dimensional shape. In some embodiments, the preformed decellularized smECM can be removed from the mold without additional processing and retain the predetermined three-dimensional shape. In some embodiments, such molded compositions having a predetermined three-dimensional shape, are reshaped (e.g., trimmed, carved) into a different desired shape. Such reshaping may occur prior to or at the time of use, or at some other desired time.

In some embodiments, a method for treating volumetric muscle loss (VML) is provided, including injecting in a subject with VML a therapeutically effective amount of a composition that includes at least one of decellularized smECM hydrogel and decellularized smECM-PhF hydrogel at at least one site of VML in the subject. In other embodiments, the method instead includes implanting in a subject with VML a therapeutically effective amount of a composition that includes at least one of decellularized smECM hydrogel and decellularized smECM-PhF hydrogel at at least one site of VML in the subject. In some embodiments, the implanted decellularized smECM hydrogel is pre-formed into a desired predetermined three-dimensional shape, which pre-formed hydrogel is transplanted. In some embodiments, the pre-formed shape is complementary to muscle absent due to VML, and in some embodiments, the implanted composition is of a standardized shape.

In addition to VML, the decellularized smECM may also be used, for example, as bulking agents for muscle deficiency, muscle damage, and/or muscle loss (e.g., traumatic, surgical (e.g., flaps, oncological resection), acute, or atrophy). The decellularized smECM may also be useful as contouring, sculpting or bulking agents for performing cosmetic or reconstructive surgery to treat muscle loss, damage or injury, as well as for aesthetic reasons and reconstruction and cosmetic procedures.

In a further embodiment, a commercial kit is provided comprising a composition described herein. A kit includes suitable packaging material, the composition and instructions for use. In one non-limiting embodiment, the composition includes a liquid decellularized smECM pre-gel in a vessel, maintained below its LCST. In another embodiment, the composition includes a dried (e.g., lyophilized), decellularized smECM in a vessel, an acid protease and buffer, as well as reagents for adjusting the acid protease reaction mixture to a more neutral pH (e.g., to about pH 7.4), so that an end user can make a low temperature, decellularized smECM pre-gel. The vessels may be vials, syringes, tubes or any other containers suitable for storage and transfer in commercial distribution routes of the kit.

The following examples further illustrate the present invention but should not be construed as in any way limiting its scope.

### EXAMPLES

An aim of the work performed in the examples (and associated methods) described below was to evaluate the effect of two known decellularization protocols in comparison to the novel protocol described herein on the properties and immunogenicity of hydrogels derived from porcine skeletal muscle ECM (smECM). It is described that cell and xeno-antigen-free smECM hydrogels with superior overall properties for skeletal muscle regenerative engineering and clinical indications resulted from the new decellularization method. The known decellularization protocols included detergent-based techniques (one sodium deoxycholate (SDC), the other Triton X-100), and the newly described method a mechanical disruption-based technique. Hydrogels of the decellularized smECM were fabricated from the resulting ECM. Subsequently, the effect of the different decellularization protocols on the characteristics and immunogenicity of the resulting hydrogels was examined in terms of, e.g., efficacy in DNA and xeno-antigen removal, preservation of biochemical composition, gelation kinetics, cyto-compatibility and myo-inductivity. Additionally, a xenotransplantation subcutaneous animal model was used to assess the immunogenicity of these hydrogels in vivo.

### Example 1

### smECM Decellularization and Hydrogel Formation

Since the discovery that decellularized ECM (dECM) materials could be solubilized and further processed to form hydrogels, hydrogels have been developed from dECMs of various tissues, including heart, lung, pancreas, skin, urinary bladder, bone, blood vessels, brain, fat, and nerves. However, due to tissue-specific variations in ECM composition, tissue-specific ECM-derived hydrogels can direct better physiologically relevant cellular responses over non-matched tissue sources. For skeletal muscle regenerative engineering and clinical indications, therefore, it is preferable not only for the hydrogel to be derived from skeletal muscle ECM but that a decellularization protocol is used that achieves maximal cell removal while retaining intrinsic, tissue-specific cues (e.g., growth factors).

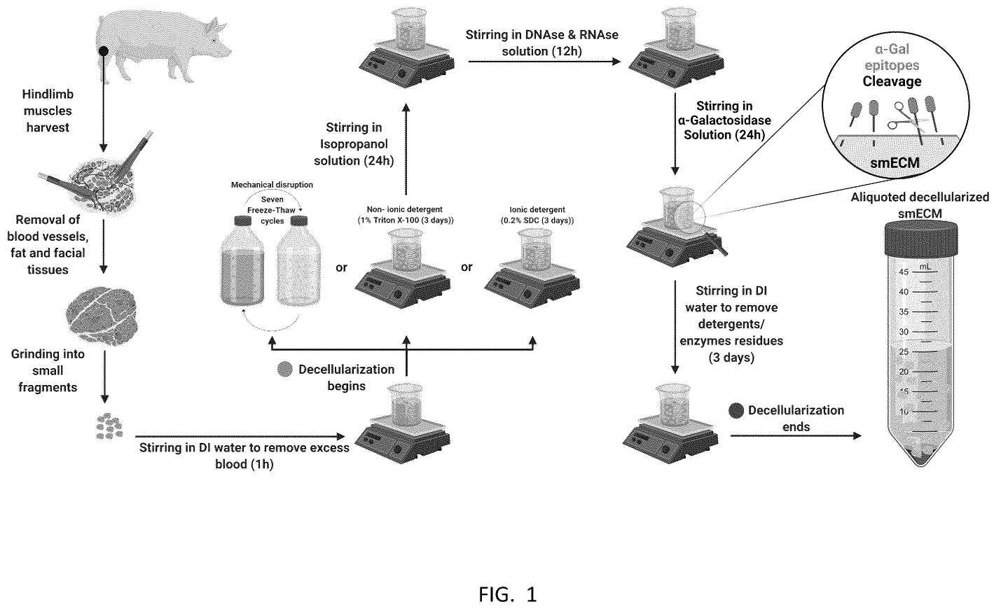

Since various tissues differ in their compositional properties, it follows that a decellularization protocol is tissue-specific, efficiently removing the cellular component while preserving the ECM molecules relevant for the intended use. For example, while tissues such as cartilage and bone can undergo relatively harsh treatment protocols, other tissues like lungs and brain require more sensitive decellularization methods to preserve their ECM composition. It is described herein that porcine smECM was decellularized following three different decellularization protocols (one newly described herein and two known in the art), each possessing a different mechanism of action (FIG. 1; also see Method 1). Unlike the known second and third protocols, the first protocol was devoid of any detergents, utilizing mechanical disruption of the ECM through exposure to multiple freeze-thaw cycles, which created ice crystals that disrupted the cell membrane leading to cell lysis. The second protocol used Triton X-100 for decellularization-a non-ionic detergent that disrupts the cell cytoplasm causing all the intracellular components (e.g., DNA and intracellular organelles) to get released from the cell. Finally, in the third protocols, the muscle tissue was treated with sodium deoxycholate (SDC)—an ionic detergent that works by disrupting the ECM causing the cells to get detached from the matrix.

Following decellularization, muscle tissues from the different protocols were incubated with isopropyl alcohol to eliminate any residual lipids in the sample that could potentially interfere with the gelation process. In addition to the isopropanol treatment, the decellularized muscle tissues were also treated with deoxyribonuclease I (DNase) and ribonuclease (RNase), which catalyze the hydrolysis of DNA and RNA, thus, further increasing the decellularization efficiency.

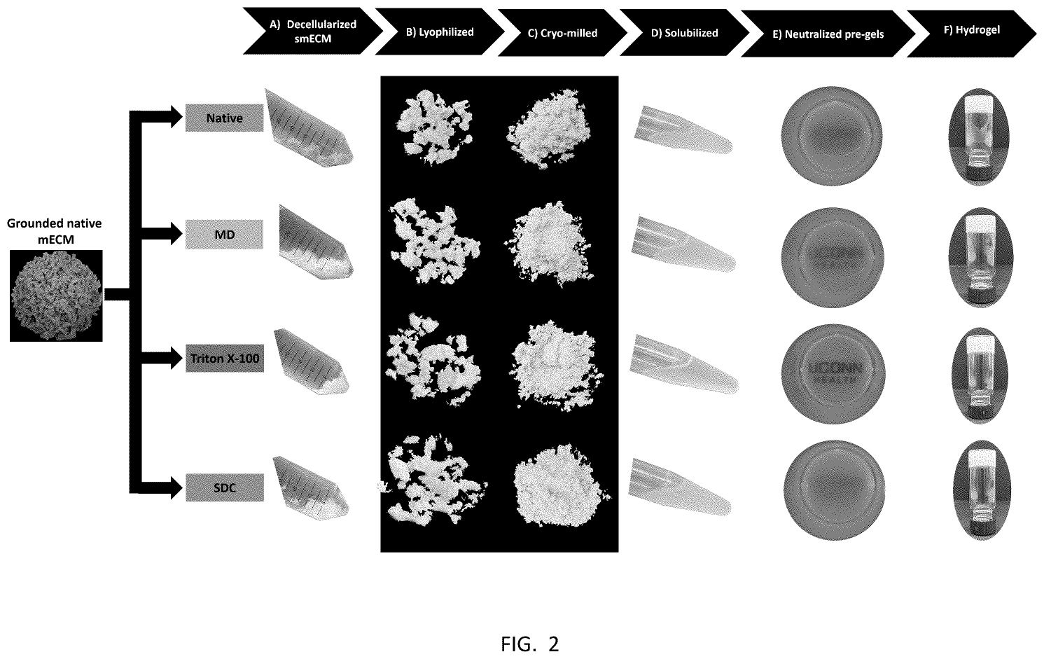

Unlike simple mincing using a pair of scalpels, the muscle tissue was ground two times using a manual meat grinder prior to decellularization (FIG. 2), to minimize the size of the muscle tissue and increase the chance for cell detachment and removal as well as the action area of the decellularization detergents and other reagents.

Post-decellularization, all tissues exhibited a white appearance, which morphologically confirms the removal of the cellular component (FIG. 2; column A). In contrast, the native non-decellularized control (e.g., hydrogels from native skeletal muscles) appeared red. The decellularized tissues from the different protocols were then freeze-dried, cryo-milled into powder, and subjected to solubilization in an acidic-based pepsin solution resulting in the formation of homogenous and viscous solutions (FIG. 2; columns B, C, and D). Post-solubilization, the pH was neutralized to ˜7.4, forming pre-gels with varying transparency (FIG. 2, column E). Subsequently, the pre-gels were incubated at 37° C. to induce gelation. Regardless of the decellularization protocol followed, all pre-gels were able to form hydrogels (FIG. 2, column F; also see Method 2).

The decellularization process can significantly alter the composition of the ECM, which can influence hydrogel characteristics and the behavior of cells encapsulated therein, all characterizations were performed on the finally-formed hydrogels. For this purpose, the resultant hydrogels were prepared at a similar final ECM concentration of 4 mg/mL to solely examine the effect of the decellularization protocol on their overall characteristics.

### Example 2

### dsDNA and Xeno-Antigen Removal Confirmation

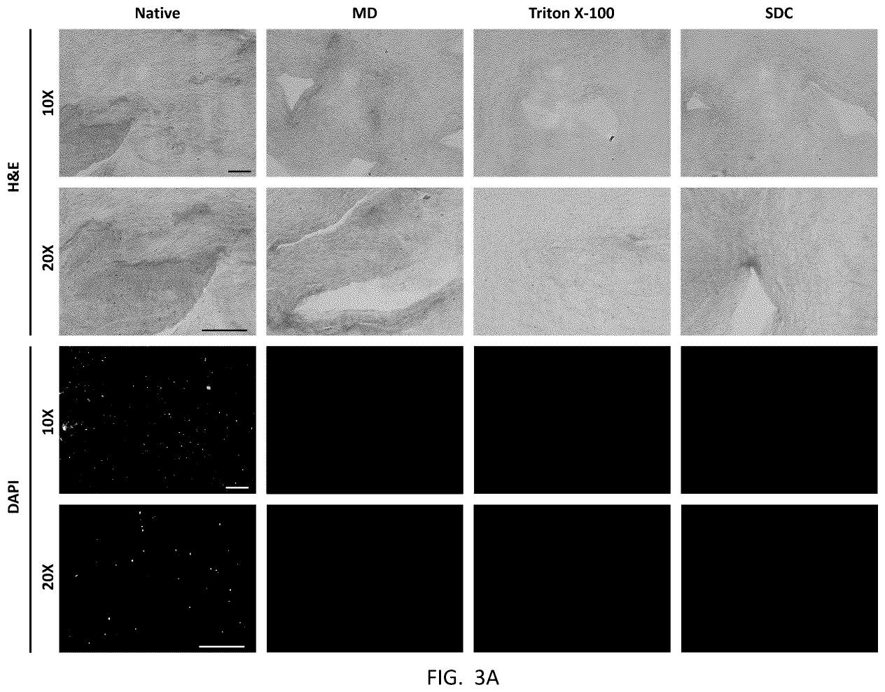

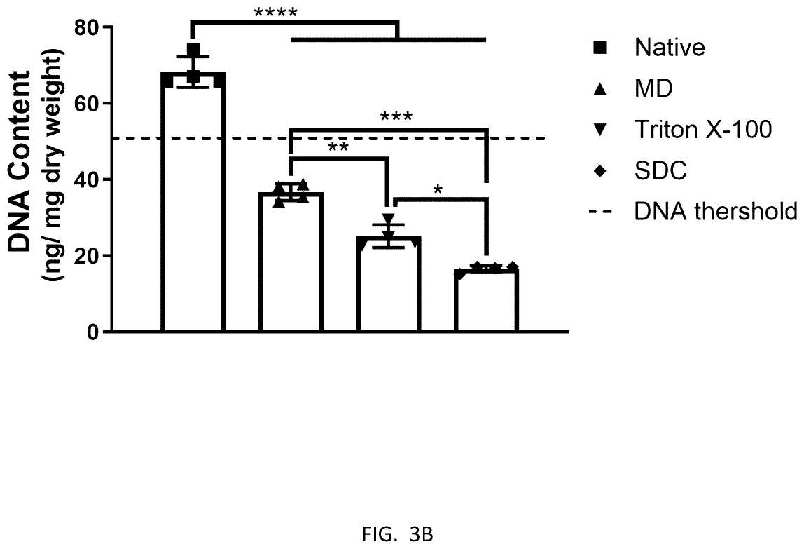

Hydrogel materials derived from porcine skeletal muscle must be thoroughly decellularized to ensure biological safety and prevent immunological responses in non-porcine transplantations. Therefore, the efficacy of different decellularization protocols in terms of DNA removal was evaluated using PicoGreen. DNA quantification confirmed that all decellularization protocols significantly reduced the total DNA content in the resultant smECM hydrogels compared to the native non-decellularized control (FIG. 3A). The DNA content in the decellularized smECM hydrogels was also below the threshold of 50 ng/mg of dry ECM, suggesting the efficacy of all decellularization protocols in removing the cellular component. Histological examinations validated these results. Staining with hematoxylin and Eosin-Y (H&E) and 4′,6-diamidino-2-phenylindole (DAPI) reaffirmed the significant reduction of nuclei after decellularization in all groups compared to the native non-decellularized control (FIG. 3B).

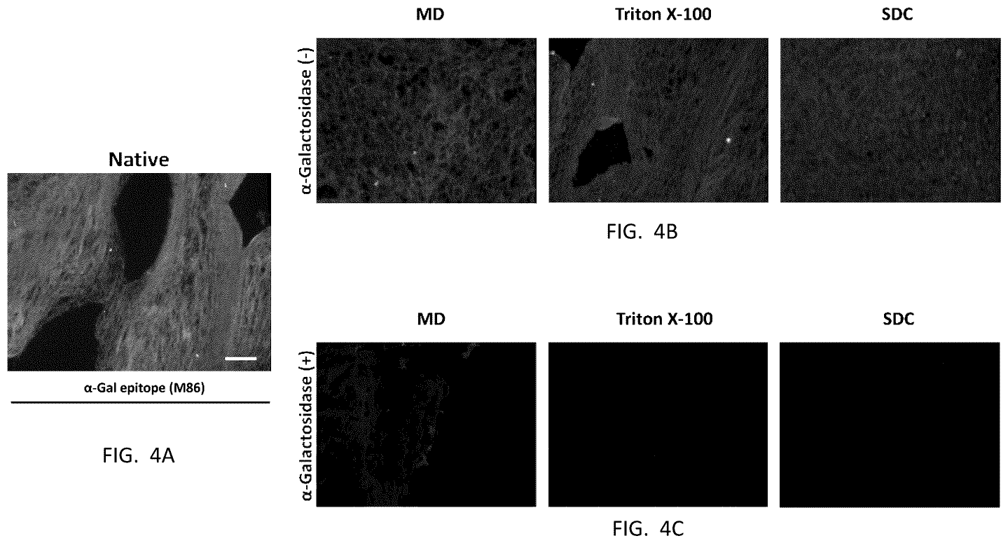

While all the tested protocols resulted in the efficient removal of the cellular component, decellularized animal grafts can still elect adverse immunological responses when transplanted in humans due to the presence of other immunogenic clements, such as xeno-antigens. The α-Gal antigen (α1,3 Gal epitopes) is the primary xeno-antigen that causes hyperacute rejection of animal ECM-derived materials in humans, and even remnant α-Gal antigen in animal ECM-derived materials clicits adverse immunological responses in the human body, negatively affecting the outcome of tissue remodeling. All non-primate mammals, including pigs, mice, rats, dogs, bovine, rabbits, horses, prosimians, and New World monkeys, carry the α-Gal antigen in their tissues. To further reduce the immunogenicity of materials derived from decellularized animal matrices, an effective decellularization protocol should remove the α-Gal epitopes from the ECM. In this context, the efficacy of the different decellularization protocols in removing the α-Gal epitopes in the finally-formed hydrogels was examined. Histological sections from each hydrogel group were stained via immunohistochemistry (IHC) with the M86 antibody, which specifically binds to the α-Gal antigen. High expression to the M86 antibody was detected in the native non-decellularized control group, confirming the presence of the α-Gal antigen in the animal ECM-derived hydrogel (FIG. 4A). Although much lower in expression intensity, the α-Gal antigen was still detected in all decellularized hydrogel groups (FIG. 4B), indicating that decellularization alone is not enough to remove the α-Gal antigen.

To further reduce the immunogenicity of the resultant hydrogels, the decellularized tissues from all groups were subjected to enzymatic treatment with α-galactosidase, which specifically cleaves the α-Gal antigen from tissues without masking their regenerative potential. This enzymatic treatment efficiently cleaved the α-Gal epitope from the resultant hydrogels in all groups based on the negative expression to the M86 antibody post-treatment (FIG. 4C). These data indicate that decellularization should be followed by an α-galactosidase treatment for efficient cleavage of the α-Gal epitope from xeno-tissues. The same treatment could be applied to animal tissues of other origins to further reduce their immunogenicity post-decellularization for potential clinical acceptance.

### Example 3

### Biochemical Characterizations

A significant benefit of using dECM-derived hydrogels is the presence of multiple ECM components that may not be present in other natural or synthetic hydrogels. Therefore, the preservation of these ECM components post-decellularization is important to provide an optimal microenvironment with appropriate biochemical cues for enhanced cell-matrix interactions. The biochemical composition of the resultant hydrogels was quantitatively and qualitatively assessed to determine the effects of the different decellularization protocols on their final biochemical blueprint. The preservation of the growth factors and total protein contents as well as other ECM molecules known to be involved in muscle tissue differentiation, development, and structural organization, such as collagens, sulfated glycosaminoglycan (sGAG), laminin and fibronectin was a focus of the work described herein.

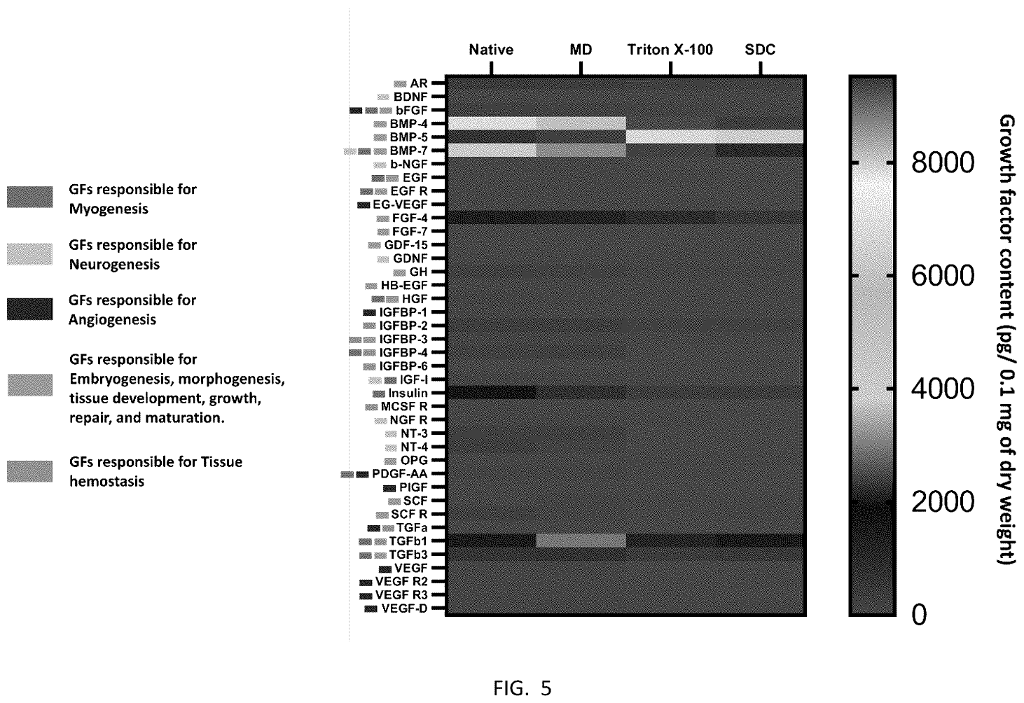

The Quantabody growth factor array was used to quantify the growth factors in the fabricated smECM hydrogels, total protein was quantified by bicinchoninic acid (BCA) assay, sGAG was quantified by dimethylmethylene blue assay, and collagen was measured by hydroxyproline quantification. A total of 38 growth factors with varying functions, including myogenesis, neurogenesis, angiogenesis, embryogenesis, morphogenesis, tissue development, growth, repair, maturation, and homeostasis, were identified in the native non-decellularized hydrogels (FIG. 5; see also Method 3). These growth factors were also identified in the hydrogels prepared by the MD method and in comparable quantities to those observed in the native non-decellularized control. In contrast, the growth factors were completely denatured or much lower in the hydrogels prepared by the Triton X-100 and SDC protocols.

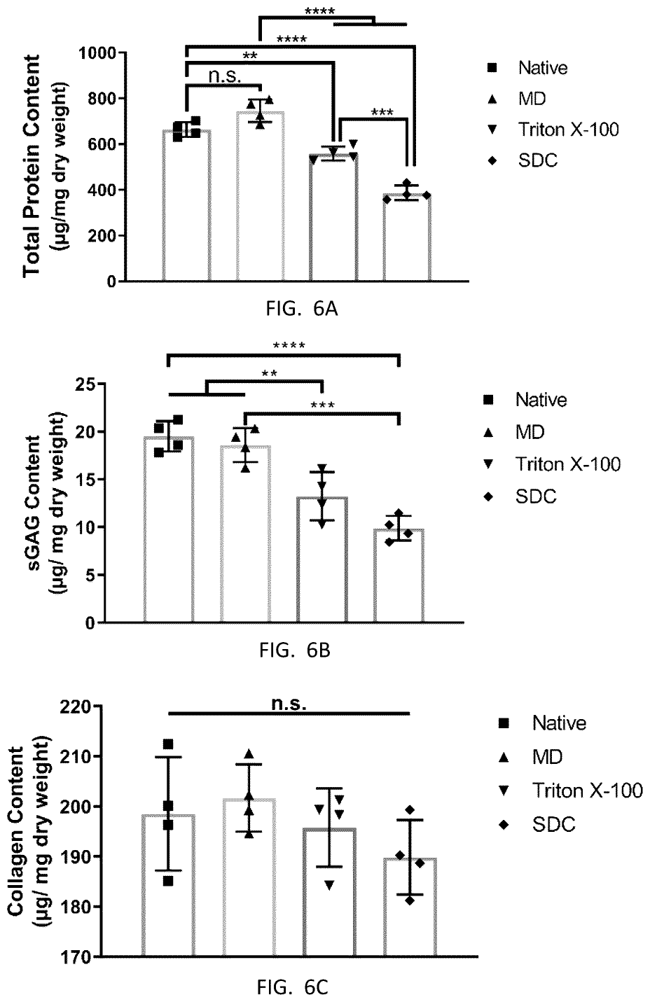

The total protein and sGAG contents were maintained at similar levels to the native non-decellularized control when decellularization was performed using the MD protocol (FIG. 6A and FIG. 6B; see also Method 3). The Triton X-100 and SDC protocols significantly reduced the total protein and sGAG contents in the final hydrogels compared to the native and MD groups. However, the collagen content remained constant across all treatment groups with no significant differences, although it was noted to be somewhat lower in the Triton X-100 and SDC groups (FIG. 6C).

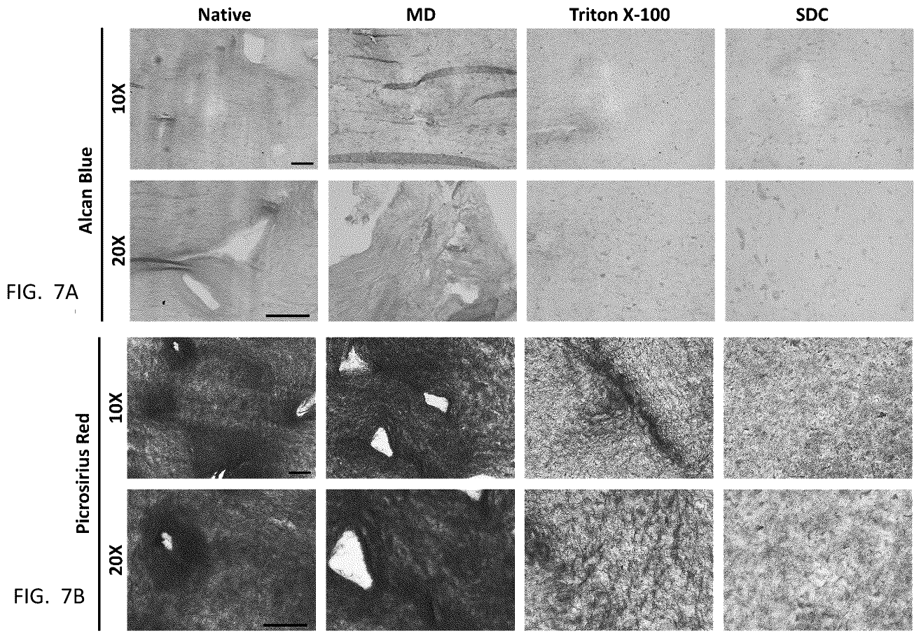

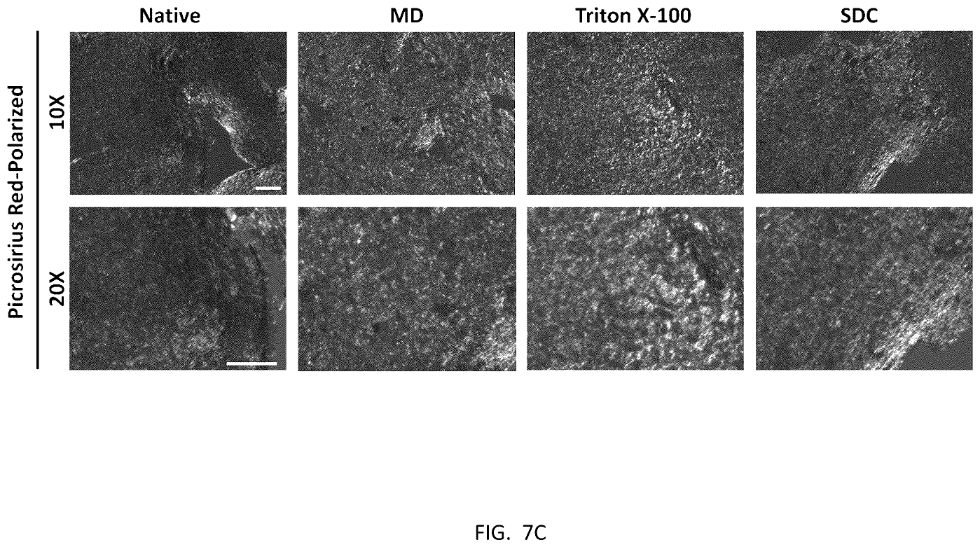

Histological examinations using alcian blue and picrosirius red staining confirmed the presence of sGAG and collagen in all hydrogels with a noticeable reduction in staining for the Triton X-100 and the SDC treated groups, which aligns with the quantification assessments (FIG. 7A and FIG. 7B; see also Method 4). Picrosirius red-polarized imaging was then performed using the same picrosirius red-stained sections to further understand the effect of the different decellularization protocols on the nativity of collagen in the formed hydrogels. This type of imaging highlights the natural birefringence of collagen fibers when exposed to polarized light. The results from the birefringence enable the precise identification of the collagen phenotype in the tissue section, where type I collagen fibers appear bright and red-yellow, in sharp contrast to type III collagen, which appears green⁵⁵. Collagen type I was more predominate than type III in the native non-decellularized hydrogels (FIG. 7C), which mimics their ratio in the native skeletal muscle tissue. The MD hydrogels contained approximately an equal amount of both collagen types. Hydrogels prepared by the Triton X-100 and SDC methods had more collagen type III with a minor presence of collagen type I, which is consistent with prior observations that both Triton X-100 and SDC can cause alteration to the nativity of collagen when used to decellularize other tissues.

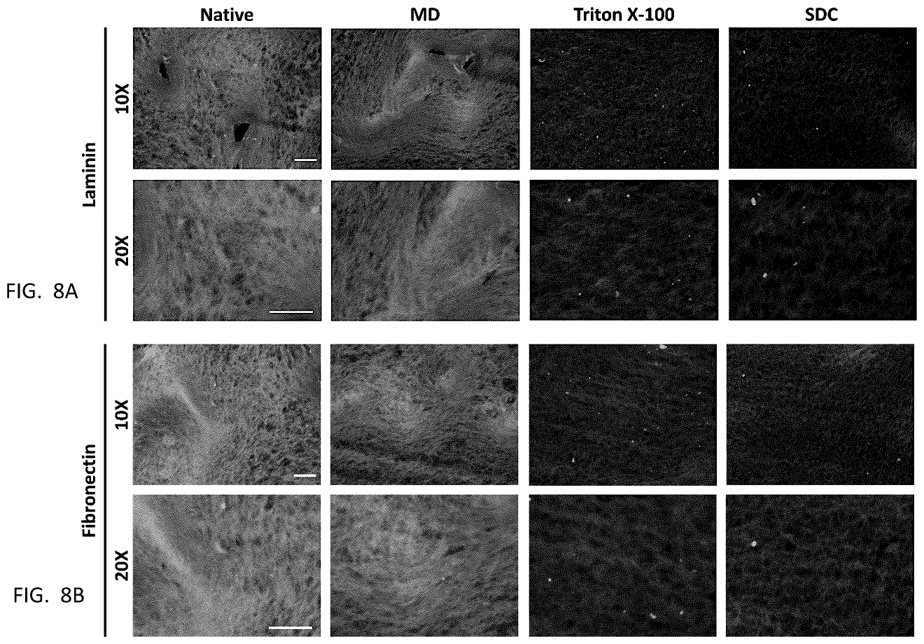

Further analysis of the biochemical composition of the smECM hydrogels was performed using IHC against two important smECM proteins, namely, laminin and fibronectin. IHC staining confirmed the presence of both laminin and fibronectin in all hydrogels (FIG. 8A and FIG. 8B; see also Method 4). However, a noticeable reduction in the expression of these proteins was observed only in the Triton X-100 and SDC-treated groups. Taken together, these results indicated that hydrogels prepared by the MD protocol retain structural ECM components that are abundant in the native skeletal muscle, including collagens, sGAG, laminin, and fibronectin, as well as non-structural ECM proteins (e.g., growth factors) that can support muscle differentiation and development. The MD decellularization protocol produced smECM hydrogels with a well-preserved biochemical blueprint compared to the Triton X-100 and SDC protocols.

### Example 4

### Gelation Kinetics

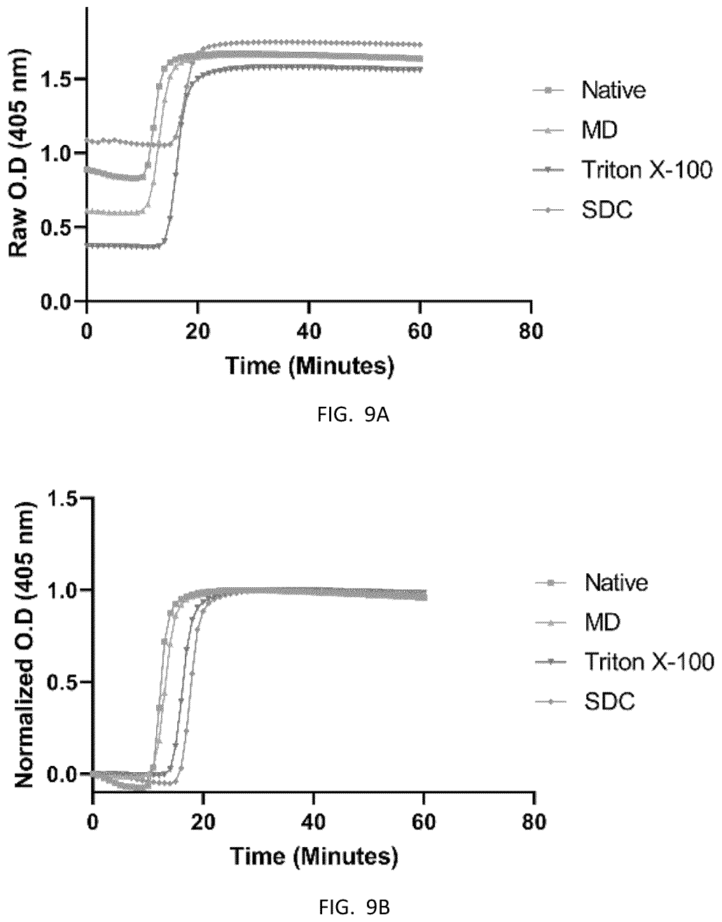

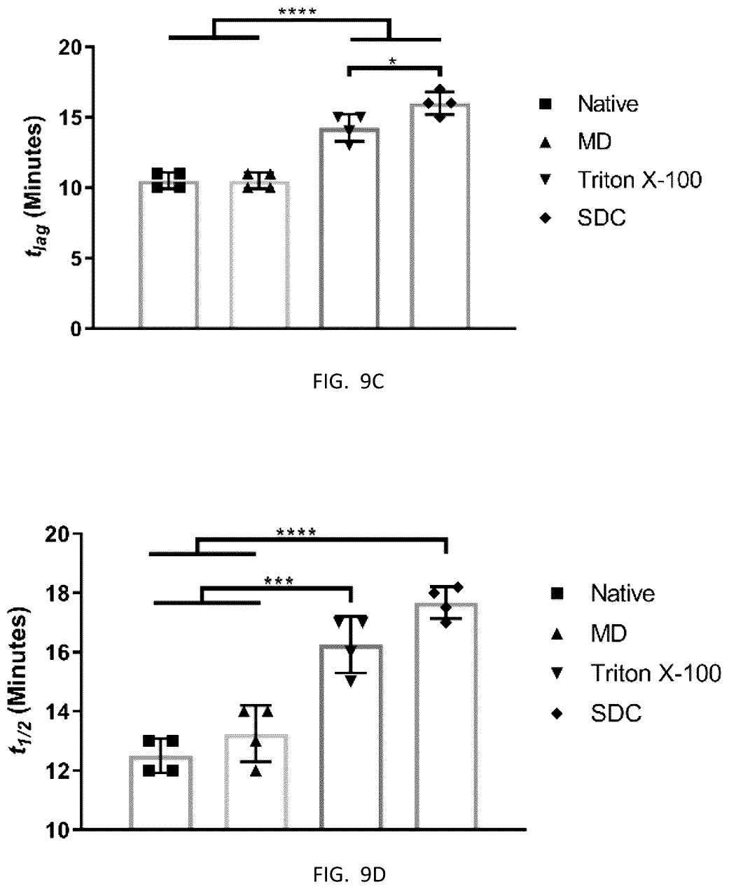

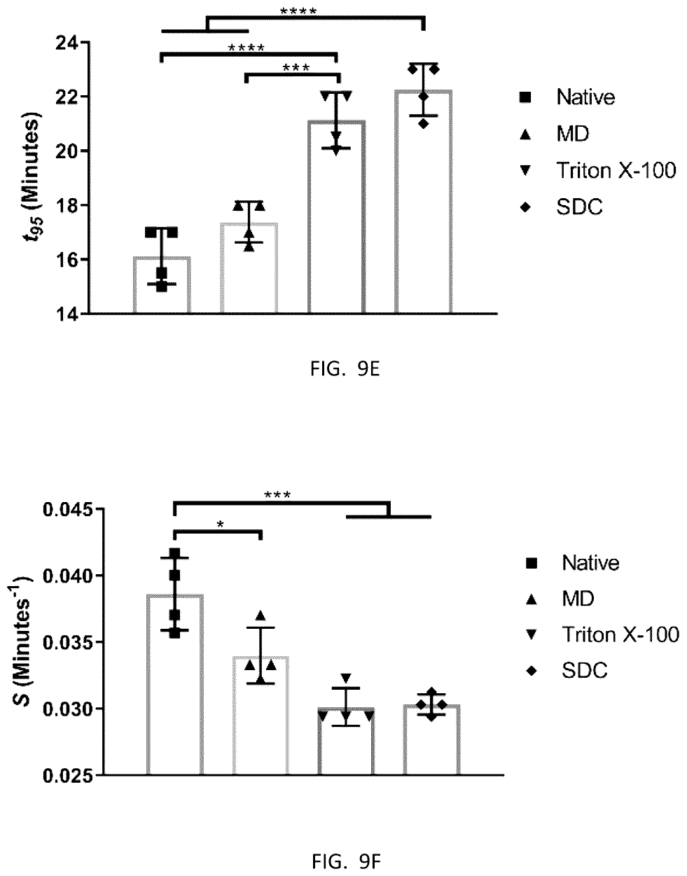

Fast hydrogel gelation is often desired for in vitro and in vivo regenerative engineering applications. This is also true for cell-populated hydrogels as rapid gelation prevents the occurrence of gravity-driven cell sedimentation that can ultimately result in an uneven cell distribution within a hydrogel matrix and limits cell loss from the site of application. Thus, the gelation kinetics of the different smECM hydrogels were analyzed spectrophotometrically via turbidimetric assessment to evaluate the impact of the different decellularization protocols on their overall gelation speed. This technique is based on the increase in turbidity and, thus, absorbance experienced during collagen self-assembly (FIG. 9A and FIG. 9B; see also Method 5). All hydrogels presented a sigmoidal profile and gelled after a lag period (t_{lag}) (FIG. 9C). Compared to the native non-decellularized control, only Triton X-100 and SDC yielded hydrogels that started gelling after a significantly longer lag phase (t_{lag} 10.5±0.5, 14.25±0.82, and 16±0.70 minutes, respectively). In contrast, the MD hydrogels had a relatively similar lag phase to that observed in the native control without any significant differences (t_{lag} 10.6±0.74 minutes). Further turbidimetric analysis revealed that the Triton X-100 and SDC treated hydrogels had significantly longer t_{1/2} (50% gelation) and t₉₅ (95% gelation) values than the native control and MD hydrogel groups (FIG. 9D and FIG. 9E). The gelation velocity was higher in the MD hydrogel group in comparison to the Triton X-100 and SDC hydrogel groups (FIG. 9F). However, no significant differences were found between the groups. Compared to the native hydrogel group, significantly lower gelation velocities were observed in the MD (*P<0.05) and Triton X-100 and SDC hydrogel groups (*P<0.001). Together, these data indicate that, unexpectedly, the MD decellularization protocol produced hydrogels with improved overall gelation kinetics.

Rheological evaluations of the different smECM hydrogels were also performed. MD hydrogels displayed comparable storage (G′) and loss (G″) modulus to the native hydrogels; whereas, Triton X-100 and SDC hydrogels showed lower G′ and G″ values (data not shown). Regardless of the decellularization protocol followed, all hydrogels showed shear-thinning properties at increasing share rates, indicating suitability for injection (data not shown).

Not to be bound by theory, but the superior gelation kinetics seen in the hydrogels produced by the MD protocol is likely due to the preservation of smECM components known to facilitate the self-assembly process of collagen molecules. dECM hydrogels are formed through a self-assembly of collagen molecules, partially controlled by the preservation of glycol proteoglycans, proteoglycans, and other ECM components. Glycol proteoglycans act as bridging molecules for the assembly of collagen fibrils, and proteoglycans regulate the collagen self-assembly process. In addition, glycoproteins like fibronectin facilitate the organization of collagen fibrils by acting as a bridging molecule during collagen self-assembly. Although no significant differences were observed when collagen content was quantified across the groups (FIG. 6C), the Triton X-100 and SDC treatments significantly reduced other components that are key regulators of the collagen self-assembly process. Thus, the preservation of collagen and other components, such as GAGs, proteoglycans, and fibronectin in the MD hydrogels, likely facilitated their faster gelation. Moreover, the presence of detergent remnants may have caused the increased gelation time in the Triton X-100 and SDC hydrogels.

### Example 5

### Ultra-Structure

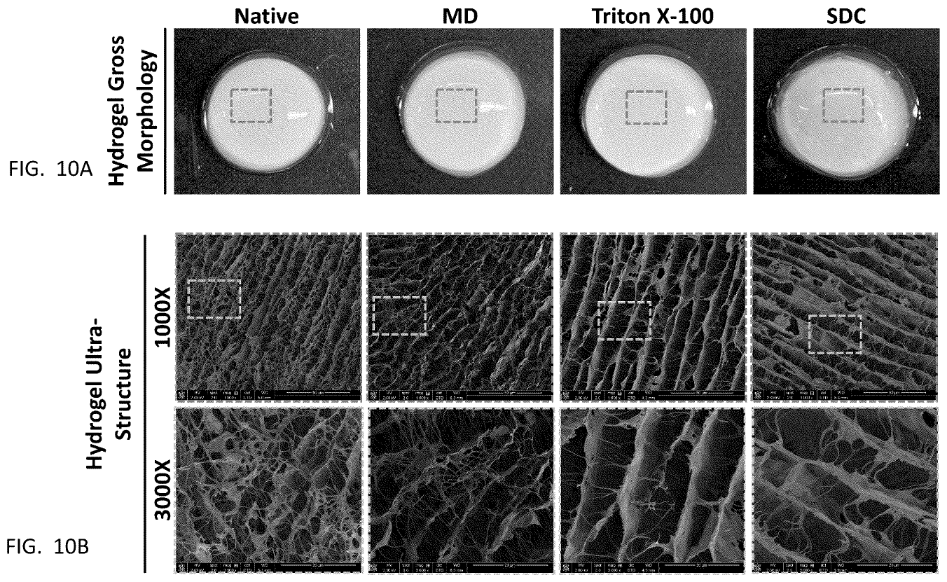

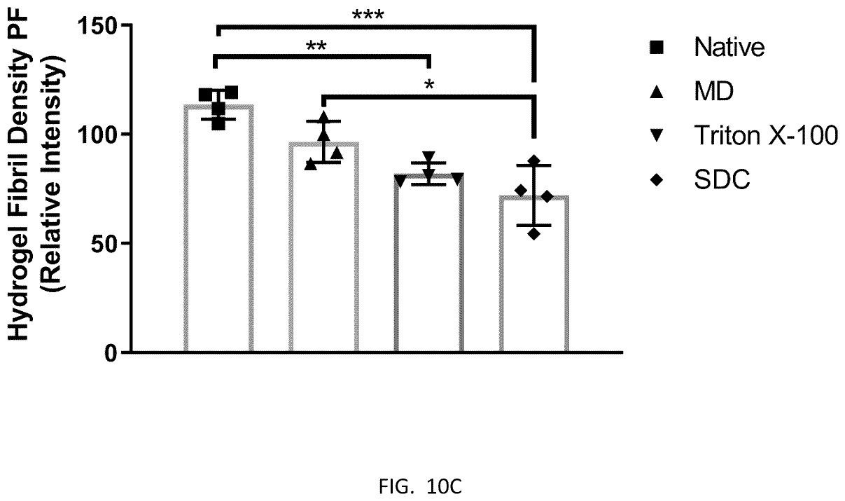

The different hydrogels (FIG. 10A) were examined under scanning electron microscopy (SEM) to evaluate their overall ultra-structure and the impact of the different decellularization protocols on their physical properties, such as fibril density. SEM images confirmed that all hydrogels were highly porous, which is advantageous to allow for efficient oxygen and nutrient transport throughout the matrix (FIG. 10B). Also, all hydrogels displayed a similar ultra-structure characterized by an array of sheet-like structures with some spacing in between. The spaces between the sheets were observed to be filled with tiny fibrils. The density of the fibrils filling the spaces between the sheets decreased in the Triton X-100 and the SDC groups compared to the native control and MD hydrogels. Quantitative analysis confirmed that the hydrogel fibril density was significantly higher in the native hydrogels compared to hydrogels produced via Triton X-100 and SDC treatments (FIG. 10C). In contrast, no significant differences were found between the MD and native hydrogels, indicating well-preserved physical properties in the hydrogels produced via MD.

Similar gelation parameters such as temperature, ionic strength, pH and ECM concentration were used to induce the gelation of all hydrogels. A temperature of 37° C. was used to form the hydrogels as lower temperatures such as 4° C. and 22°° C. are known to inhibit or prevent gelation. An ionic strength of 0.5× PBS was also used when diluting the stock to a final concentration of 4 mg/mL. Lastly, a pH of about 7.4 was used as a neutral pH is known to catalyze the collagen self-assembly process faster than significantly more acidic or alkaline pH values. As all hydrogels were fabricated following the same parameters, the significant reduction in the hydrogel fibril density observed in the Triton X-100 and SDC groups is attributed to depletion of ECM molecules in these hydrogels that promote self-assembly, which may have attenuated the degree of collagen self-assembly and attenuated the resultant fibril density.

### Example 6

### Migration

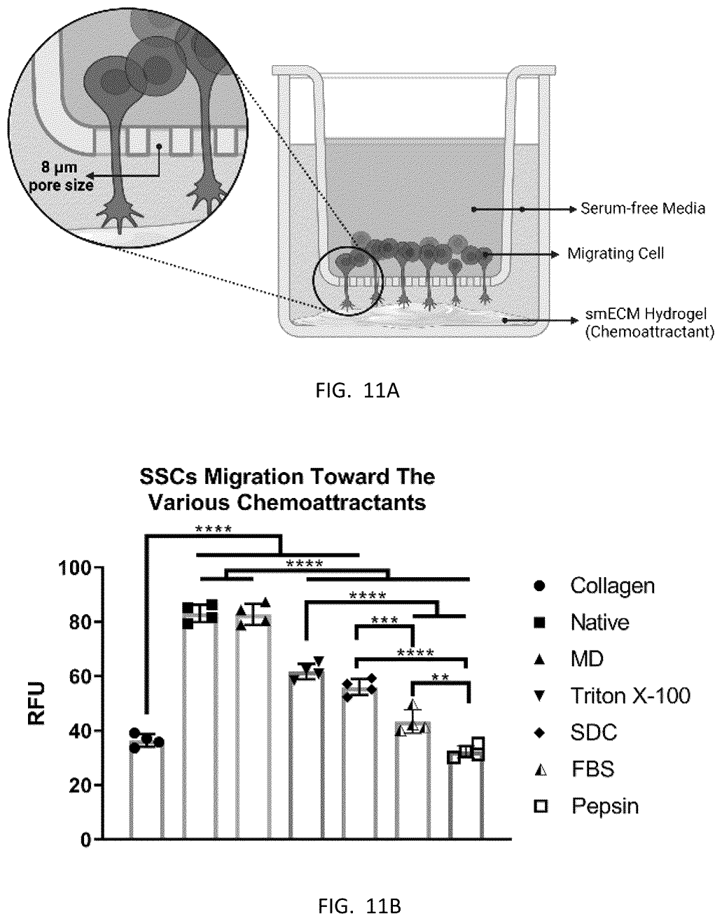

Recruitment of resident muscle progenitor cells (e.g., SSCs) into an injury site can accelerate the repair process. It is known that decellularized smECM scaffolds can act as a recruiting milieu for SSCs due to the presence of preserved growth factors involved in guiding cell recruitment such as basic fibroblast growth factor (bFGF), bone morphogenetic proteins-7 (BMP-7), epidermal growth factor (EGF), epidermal growth factor-r (EGF-R), hepatocyte growth factor (HGF), insulin-like growth factor binding protein-4 (IGFBP-4), insulin-like growth factor-i (IGF-I), platelet-derived growth factor-aa (PDGF-AA), transforming growth factor beta-1 (TGFb1), and transforming growth factor beta-3 (TGFb3). These growth factors were also identified in certain smECM hydrogels described herein, suggesting their preservation. Whether smECM hydrogels can act as chemoattractants for SSC migration and whether the decellularization protocol can influence these chemoattractive properties was investigated. To do so, a trans-well-based cell migration assay was conducted (see Method 8, below), where primary SSCs were cultivated on the top porous membrane of the trans-well system, while the different hydrogels were dispensed at its lower compartment (FIG. 11A). in addition to evaluating the different smECM hydrogels, 10% FBS, and 1 mg/mL pepsin in 30 mg/mL acetic acid solution in H₂O, were added as positive and negative controls, respectively. Further, commercially available collagen type I hydrogel was added as a third control for comparison. Regardless of the decellularization protocol, SSCs showed significantly higher migration toward all smECM hydrogels in comparison to all controls, including the commercially available collagen (FIG. 11B). However, SSC migration was significantly higher toward native and MD hydrogels in comparison to the hydrogels produced by the Triton X-100 and SDC protocols. No significant differences were observed in the magnitude of SSC migration toward the native and MD hydrogels. The diminished SSCs migration toward the Triton X-100 and SDC hydrogels compared to the native and MD may be ascribed to the poor preservation of growth factors in these hydrogels due to the harsh decellularization protocols applied to prepare them, rendering them less effective chemoattractants. These data highlight the chemoattractive properties of smECM hydrogel, generally, over synthetic analogs and demonstrate the superior, native hydrogel-like properties of the mechanical disruption decellularization protocol in comparison to known decellularization methods.

### Example 7

### Cyto-Compatibility

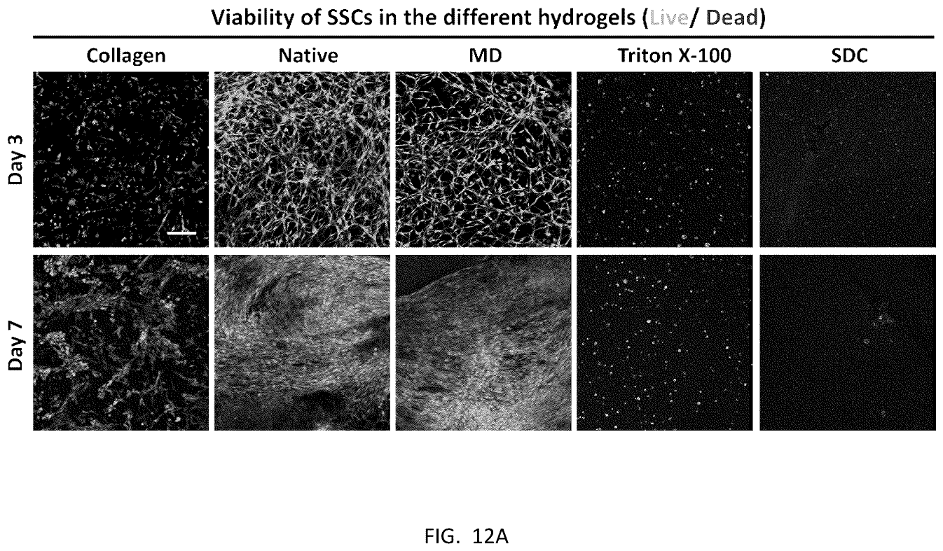

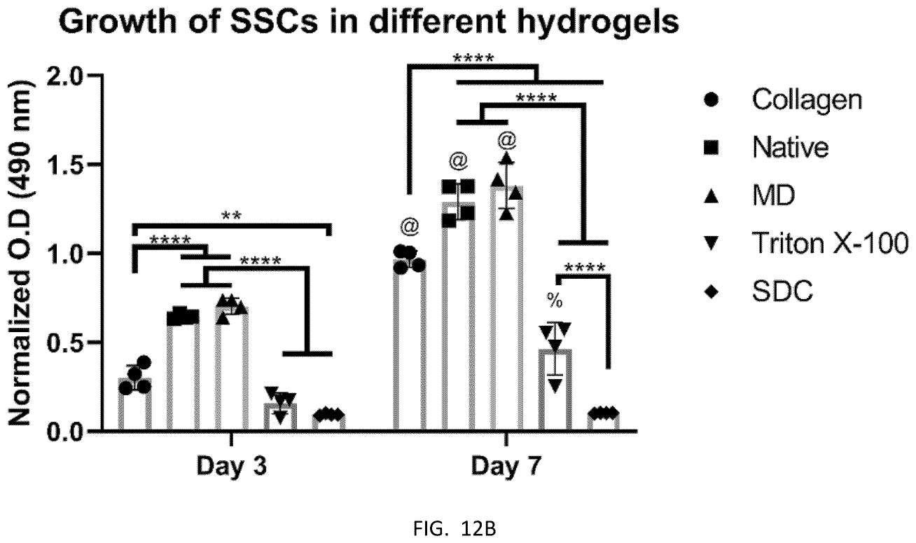

The impact of the decellularization protocol on the cyto-compatibility of the finally-formed hydrogels was investigated. This was done by embedding primary SSCs within the different hydrogels, followed by assessing their viability and growth over time (see Method 9). Collagen type I hydrogel was added as a control in all biological evaluations for comparison as it is known for cyto-compatibility and has been widely utilized as a microenvironment for growth of SSCs. Live/dead viability confocal images showed that the cells were highly viable within the collagen, native, and MD hydrogel groups at both culture timepoints (3 and 7 days) and adopted a spindle morphology with multiple processes, indicating good adhesion to the fibrils (FIG. 12A). In contrast, and unexpectedly, the Triton X-100 and the SDC rendered cytotoxic hydrogels as cells were found to be completely round and dead within these hydrogels at both culture timepoints.

An MTS assay was then used to assess the growth of SSCs in the different hydrogels (see Method 9). The MTS growth assay results showed that SSCs displayed significantly higher growth rates in the native and MD hydrogel groups at both culture timepoints compared to all other hydrogels, including the commercially available collagen type I (FIG. 12B). No statistically significant differences were observed in growth rates between the native control and MD groups. These data indicate that out of all the decellularization protocols followed, only the MD method described herein produced hydrogels that are highly cyto-compatible. In addition, the cells were more metabolically active and displayed significantly higher growth rates when they were surrounded by their original ECM environment in comparison to the pure collagen type I. The smECM hydrogel as produced by the methods described herein provided the cells with an improved microenvironment over other methods, one that included various tissue-specific components that facilitated the enhancement of cell growth.

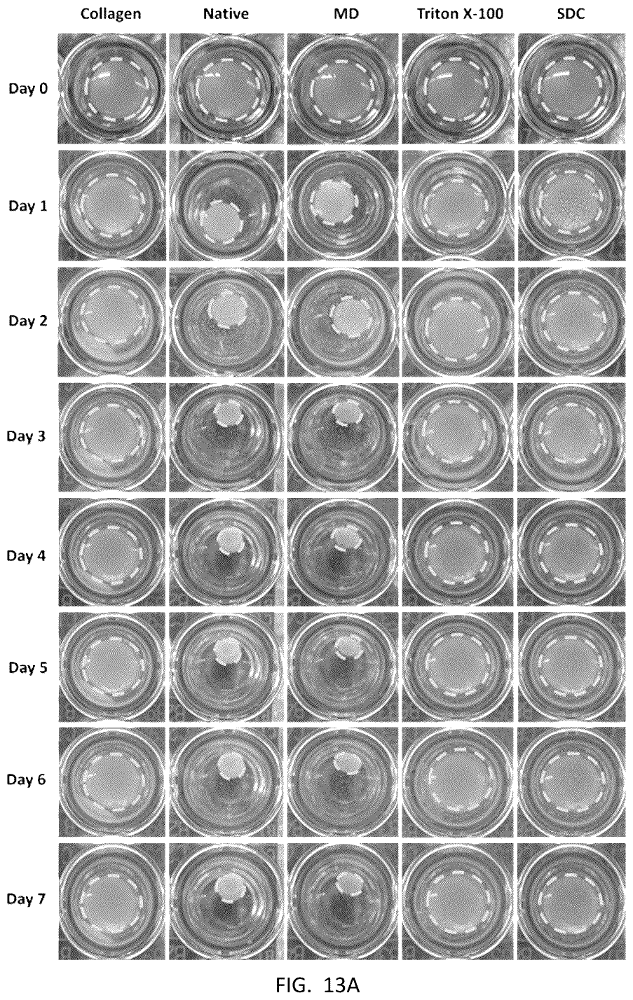

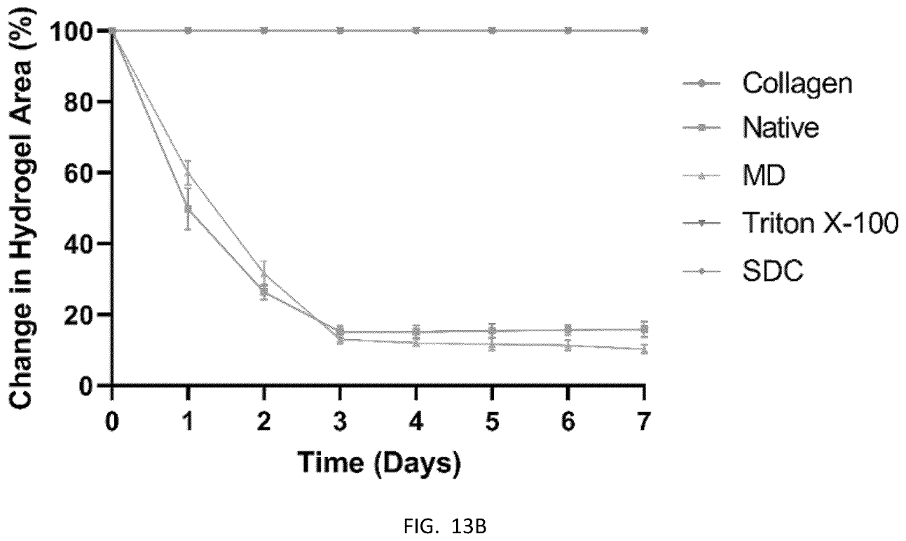

Over the cultured period, the native and MD hydrogels underwent a significant cell-mediated contraction (FIG. 13A). In contrast, when the cells died as for the Triton X-100 and SDC-decellularized ECM, the hydrogels retained their initial shape and size and did not contract. Similarly, the commercially available collagen type I hydrogel did not undergo any contraction over the course of culture. Quantification of the change in hydrogel area revealed that this cell-mediated contraction started as early as day one of culture, reaching approximately 85% contraction by day 7 in both the native and MD hydrogel groups (FIG. 13B). These findings are consistent with the Live/Dead and MTS data, and further confirm that the cells within the native and MD hydrogel groups were highly viable and metabolically active, as evidenced by their ability to attach and pull the matrix fibrils as they proliferated and migrated within their 3D space, causing them to contract. Although cells within the collagen type I hydrogel were viable, they failed to contract the matrix and did not reach the same level of metabolic activity as the native and MD hydrogels. The absence of certain important instructive tissue-specific ECM components is thought to be an important factor in the observed difference in activity and character.

The rapid cell-mediated contraction observed in the free floating MD hydrogels of the disclosure can be advantageous for bioengineering highly anisotropic muscle tissues. In skeletal muscle regenerative engineering, achieving an anisotropic cellular organization within hydrogels is necessary for producing biomimetic and highly functional engineered muscle tissues. In this context, one effective way to guide the cellular organization within hydrogels is through the concept of hydrogel tethering, where a cell-populated hydrogel is tethered from two parallel ends (between two parallel pillars) to restrict its cell-mediated contraction. This restricted contraction builds-up a uniaxial tension along the hydrogel's matrix that facilitates the rearrangement of its fibrils into aligned fibers pointing toward the direction of tension, thus guiding the alignment of the encapsulated cells toward the same direction of tension. However, for the successful generation of highly anisotropic cellular organization within tethered hydrogels, sufficient tension is required, which is dependent on the degree of hydrogel contraction. The more the hydrogel contracts, the greater the tension becomes. Hence, hydrogels that possess the ability to undergo rapid cell-mediated contraction, like the developed and described herein, can provide excellent substrates for engineering highly anisotropic muscle tissues following such an approach. In addition, smECM hydrogels as described herein may be further advantageous due to their tissue-specificity.

As an injectable, an MD hydrogel can adhere to and interact with the underlying tissue matrix and as such is not free floating, reducing the ability of cells to contract the MD smECM to such a degree as seen for free floating MD smECM.

### Example 8

### Myo-Inductivity

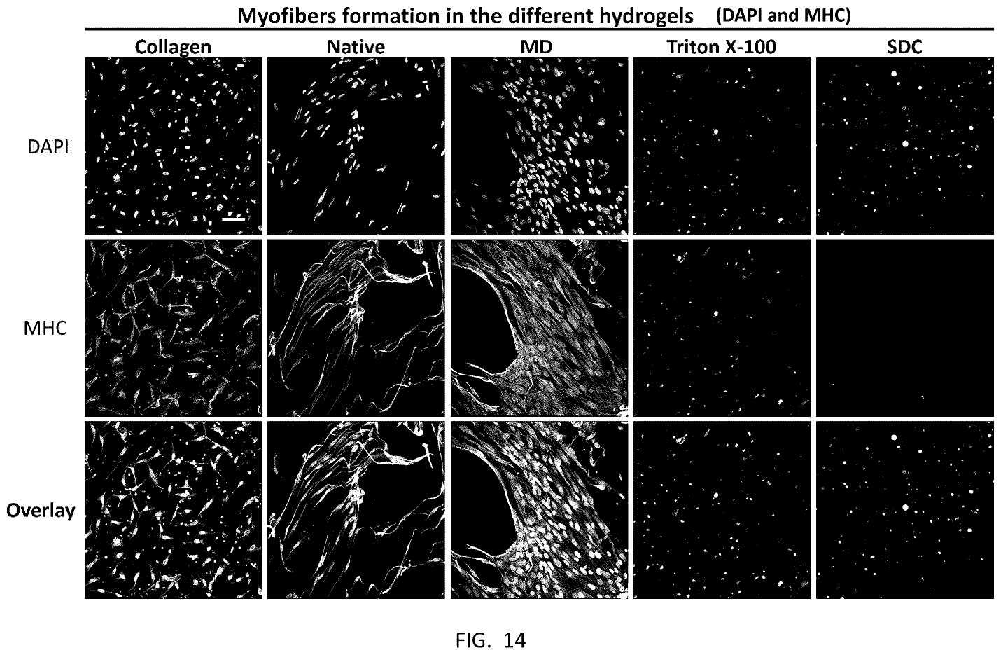

An aim of the subject matter of the present disclosure was to develop an smECM hydrogel with a tissue-specific microenvironment for enhanced myogenesis of muscle cells, one with unimpacted or minimally impacted myo-inductive cues normally present in the skeletal muscle matrix. SSCs were encapsulated within the different smECM hydrogels and their differentiation was assessed to estimate the impact each decellularization protocol on myo-inductivity. Commercially available collagen was added as a control for comparison. Immunofluorescence-stained hydrogels for myosin heavy chain (MHC) showed that SSCs could differentiate and rapidly fuse into multinucleated myofibers only in the native and MD hydrogel groups, as compared to the other groups, including the commercially available collagen (FIG. 14). In the collagen control, SSCs were differentiated into myocytes based on the positive expression of MHC, but no evidence of myofiber formation was found. The Triton X-100 hydrogels contained a small population of MHC-positive cells, whereas MHC expression was utterly negative in the SDC group, possibly due to their cytotoxicity. These observations indicate the myogenic potency of smECM hydrogel over collagen, and demonstrate the profound effects that the decellularization method can have on the myo-inductivity of the finally-formed smECM hydrogel. smECM hydrogels produced by the MD protocol described herein exhibited superior properties for, e.g., skeletal muscle regenerative engineering.

As the presently described decellularized smECM hydrogel was derived from a xeno-tissue, its biological safety needed to be tested in vivo in a suitable animal model to evaluate the immunological response and ability to integrate with the surrounding tissues.

### Example 9

### In Vivo Immunogenicity Assessments

Materials obtained from allogeneic sources avoid some concerns associated with immunogenicity, but they can only be obtained from older individuals, have large batch-batch variability, and are not readily available. In contrast, xenogeneic materials from, e.g., porcine tissues are readily available and can be produced from younger tissue sources, which is desirable for regenerative medicine therapies. However, xenogeneic materials can have potential immunogenic issues and regulatory hurdles. For these reasons, in vivo biological safety of the porcine skeletal muscle-derived hydrogels was investigated to determine the effect the decellularization protocol has on overall immunogenicity.

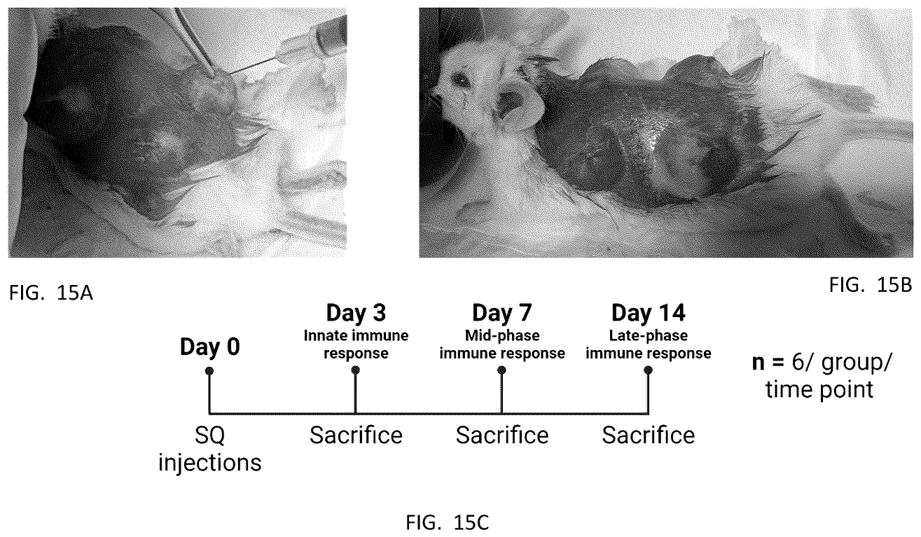

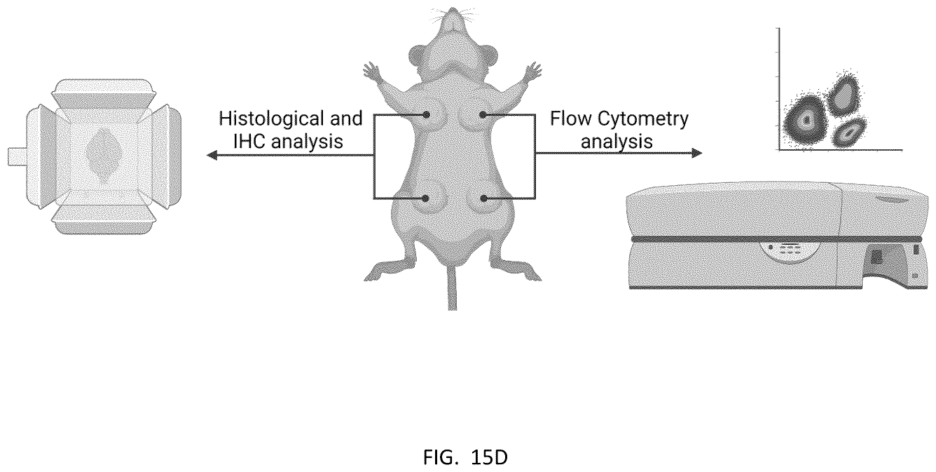

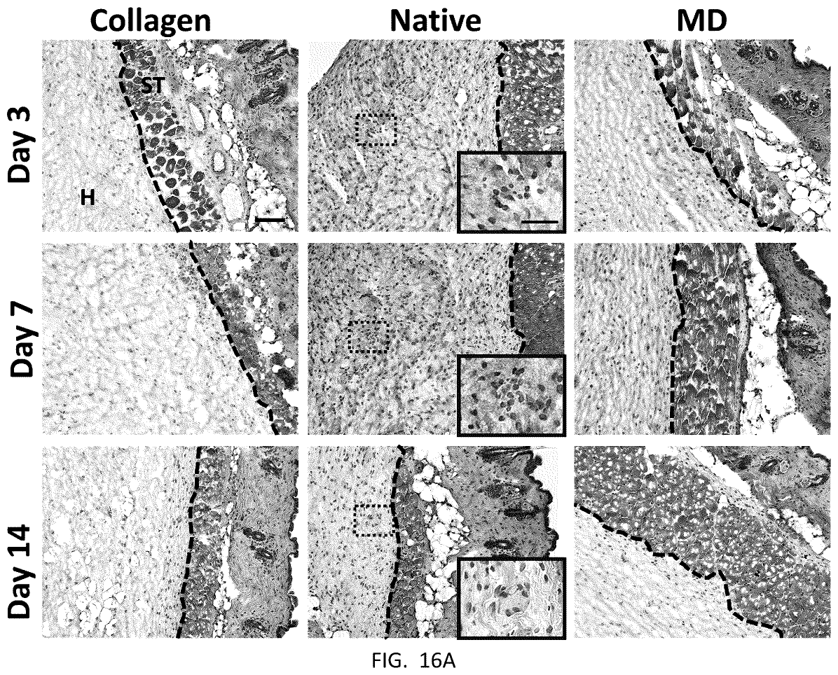

Balb/c mouse was chosen as the xenotransplantation model. This animal model is immunocompetent with a fully functioning immune system and extensively has been used for biocompatibility and immunogenicity assessments of various xenogeneic tissues. In addition to assessing the immunogenicity of the MD hydrogels, the native non-decellularized and collagen type I hydrogels were added as pro-inflammatory and pro-remodeling controls, respectively. Each hydrogel was injected subcutaneously, a common delivery route for ISO standard biocompatibility tests (FIG. 15A and FIG. 15B). The injected hydrogels were harvested for histological, IHC, and flow cytometry assessments on days 3, 7, and 14 post-injection (FIG. 15C and FIG. 15D). These timepoints were selected for assessing the early (innate), mid, and late-phase immune response, respectively, given macrophage response and polarization during this timeframe is thought to be representative of long-term biocompatibility and tissue repair. Longer time points were not investigated because ECM-derived hydrogels are known to degrade completely by 3-4 weeks in vivo.

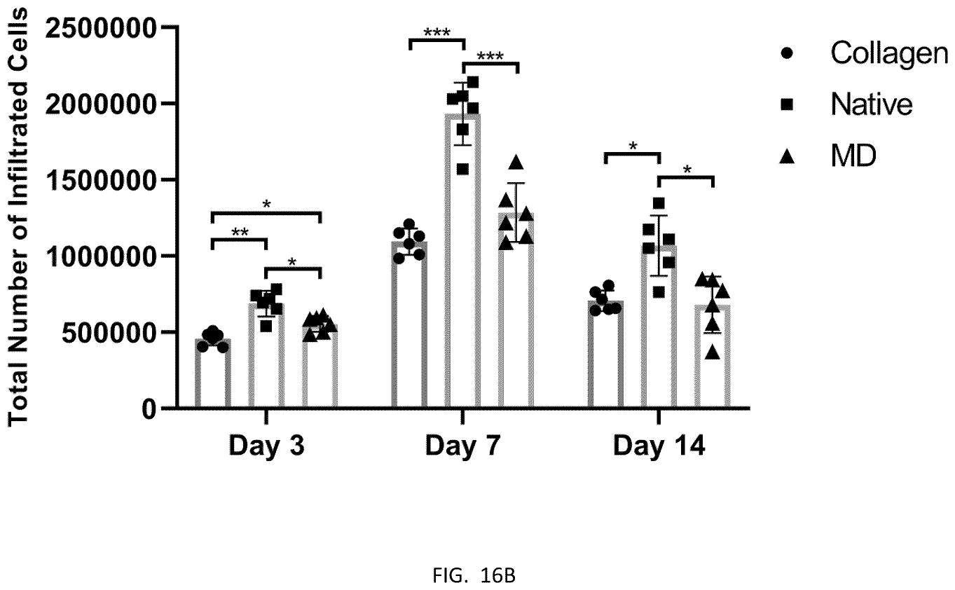

Harvested biomaterials were stained with hematoxylin and Eosin-y to assess cellular infiltration and density. Greater cellular infiltration was generally observed in the native non-decellularized hydrogels compared to the collagen type I control and MD hydrogels at all timepoints (FIG. 16A). In addition, foreign body giant immune cells (FBGIC)—a collection of fused macrophages that are generated in response to the presence of an unfavorable material, were clearly present at all timepoints in the native non-decellularized hydrogels. In contrast, fewer cellular infiltration and no FBGIC were observed in the collagen type I and MD hydrogels at all timepoints. To obtain a quantitative measure of the density of the infiltrated cells, the harvested biomaterials were digested, and the number of released cells was counted. The number of infiltrated cells was significantly higher in the native non-decellularized hydrogels compared to the collagen type I control, and MD hydrogels at all timepoints, which aligns with the hematoxylin and Eosin-y findings (FIG. 16B). The MD hydrogels had a significantly higher number of infiltrated cells at day 3 in comparison to the collagen type I control. However, no significant differences were observed between these two groups at later timepoints. The density of the infiltrated cells was found to significantly increase on day 7 compared to day 3 in all groups, reaching its peak value before it declined by day 14. This increase in the number of infiltrated cells during the mid-phase is part of the wound healing immune response.

The greater cell infiltration observed in the native non-decellularized control group suggests an unfavorable immune response to the biomaterial, as greater cell infiltration is thought to be one of the initial signs of acute graft rejection. In addition, the clear presence of FBGIC further suggests graft rejection of the native non-decellularized hydrogels. In contrast, the significantly lower number of infiltrated cells in the collagen type I control and MD hydrogels compared to the native non-decellularized control at all timepoints and the clear absence of FBGIC suggest acceptance of these biomaterials.

While assessments such as H&E staining and quantifying infiltrated cell numbers can offer insight into the biocompatibility and immunogenicity of biomaterials, they cannot provide detailed and comprehensive information regarding the host immune response. A more accurate measure for evaluating the biocompatibility and immunogenicity of biomaterials is to identify the phenotype of the infiltrated cells and assess their predominancy. Macrophages and T-cells are central immune cells that commonly invade biomaterials post-administration in vivo, regardless of biocompatibility of the biomaterial. Assessing the predominancy of the phenotype of infiltrating macrophages and T-cells can provide an indication of biomaterial rejection (inflammation) or acceptance following administration. For instance, macrophages can be polarized into M1 (pro-inflammatory) and M2 (anti-inflammatory) subtypes. The M1 macrophages are known to elicit a pro-inflammatory immune response, and their predominancy in the biomaterial is often linked to graft rejection. In contrast, M2 macrophages stimulate an anti-inflammatory immune response which mimics the immune response during wound healing and tissue remodeling.

Thus, their predominancy is often an indication of graft acceptance. Similarly, T-cells are subcategorized into T-Helper (T-Help) and T-cytotoxic (T-Cyto) subtypes. T-Help T-cells elicit a pro-remolding immune response, whereas T-Cyto T-cells elicit a pro-inflammatory immune repose. Hence, it can be speculated that the predominancy of the T-Help T-cells in the biomaterial is more favorable.

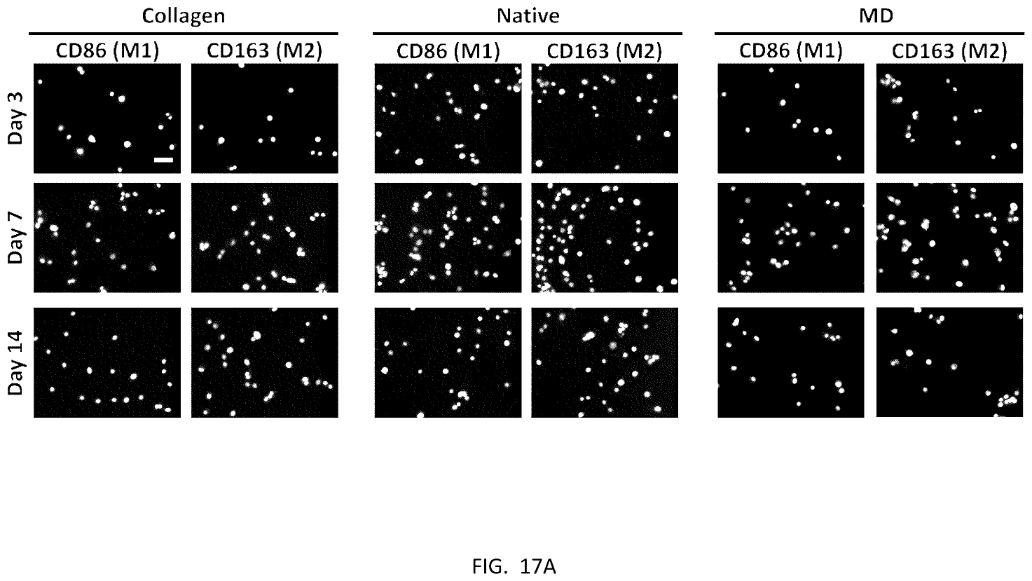

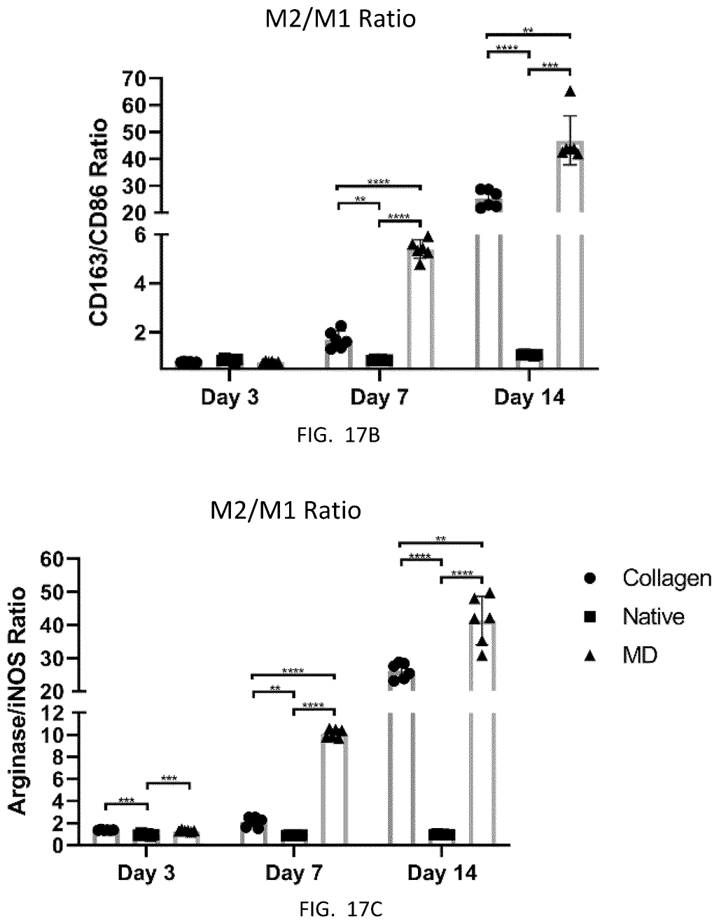

Comprehensive IHC and flow cytometry analyses were performed to distinguish the phenotype and predominancy of the infiltrated immune cells within the different hydrogels. CD86 (M1 macrophage marker) and CD163 (M2 macrophage marker) were used for the IHC and flow cytometry analysis, and the percentages of CD86+ and CD163+ cells measured by the flow cytometry assessment were used to calculate the M2/M1 ratio to estimate the magnitude of macrophage predominancy. IHC analysis indicated the presence of polarized macrophages toward both the M1 and M2 subtypes in all groups at all timepoints but in distinct densities (FIG. 17A).

At day 3, the ratio of M2/M1 macrophages for all three hydrogels was below 1, suggesting that the early-phase macrophage response was M1 dominant (FIG. 17B). At day 7, the native non-decellularized hydrogels remained M1 dominant, while the collagen type I and MD hydrogels switched to an M2 dominant response. At day 14, the native non-decellularized hydrogels continued to be M1 dominant, while the M2 predominancy further increased in the collagen type I and MD hydrogels. This demonstrated a transition from a pro-inflammatory to pro-remodeling macrophage polarization response for the collagen and MD hydrogel, which is a characteristic of wound healing and indicative of graft acceptance. However, compared to the collagen type I control, the MD hydrogels showed a more favorable immune response, as evidenced by the significantly higher values in the M2/M1 ratio analysis on days 7 and 14.

To further confirm the subtype of the polarized macrophages, flow cytometry analysis against additional M1 and M2 markers, (iNOS) and (Arginase), respectively, were also performed, and the ratio of Arginase/iNOS was calculated using the percentages of Arginase+ and iNOS+ cells measured by the flow cytometry assessment. Identical ratio tends were also observed using these additional markers, further emphasizing the above findings (FIG. 17C).

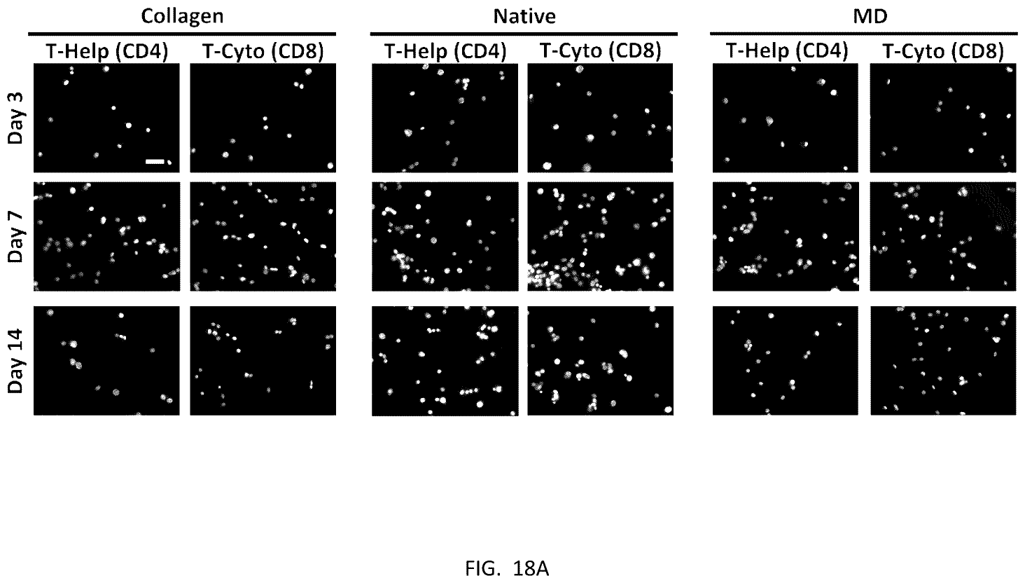

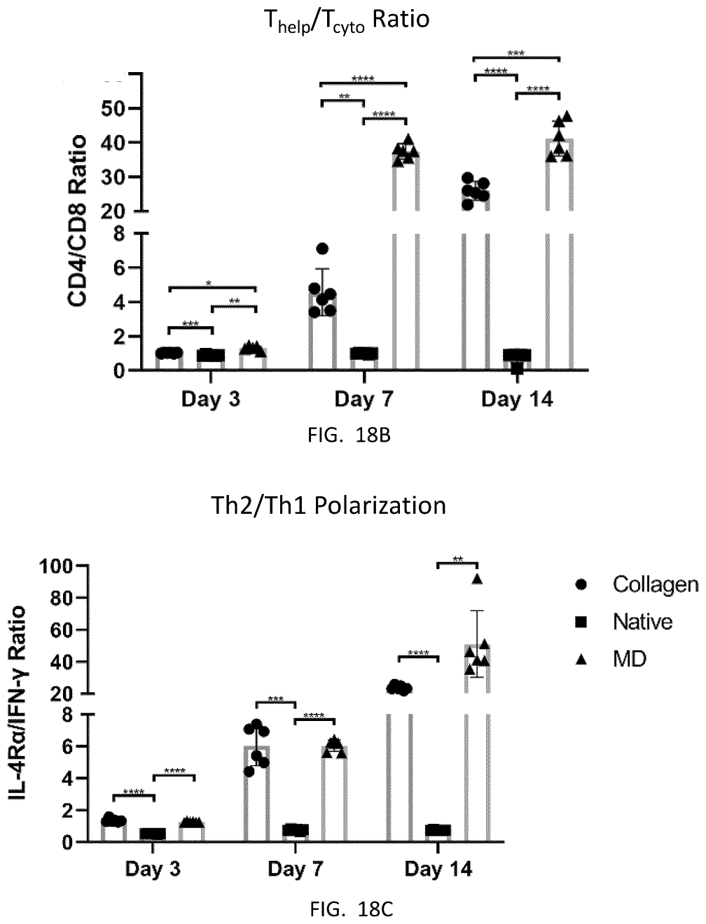

A similar assessment was performed to calculate the ratio of T-Help/T-Cyto in the different hydrogels using CD4 (T-help marker) and CD8 (T-Cyto marker). IHC analysis showed that T-Help and T-Cyto T-cells were present in the different hydrogels but in distinct densities and expression magnitude (FIG. 18A). Specifically, T-Help T-cells were found to be present at all timepoints in the different hydrogels. In contrast, T-Cyto T-cells were only present at days 3 and 7 in the collagen type I and MD hydrogels, whereas their presence was detected at all timepoints in the native non-decellularized hydrogels. Comparing the ratio of T-help to T-Cyto, the native non-decellularized hydrogels were consistently T-Cyto T-cells dominant at all timepoints (FIG. 18B). In contrast, the collagen type I and MD hydrogels were always T-Help T-cells dominant and the T-help T-cells predominancy was further increasing overtime. This transition in immune response toward the collagen type I and MD hydrogels from a lesser to a higher T-Help T-cell dominant environment suggests that only the native non-decellularized hydrogels were being rejected. However, compared to the collagen type I control, the MD hydrogels showed significantly higher values in the T-Help/T-Cyto ratio analysis at all timepoints, suggesting a more favored immune response toward the MD hydrogels.

As the immune response is a highly complex process, T-Help T-cells (CD4+) predominancy does not always indicate a favorable immune response. For instance, T-help T-cells can be polarized into Th1 and Th2 subtypes, which are both CD4+. Th1l T-cells produce a set of cytokines, including IFN-γ, IL-12, IL-18, and IL-27 which stimulate macrophage polarization toward the M1 pro-inflammatory subtype. In contrast, Th2 T-cells stimulate macrophage polarization toward the M2 pro-remodeling subtype by producing IL-4Rα, IL17RB, CCR3, and CCR4. Therefore, the polarization of macrophages toward the M1 and M2 subtypes is mainly determined by the subtype into which T-help T-cells polarize.

To distinguish the subtype of the T-helper T-cells in the different hydrogels, as well as the predominancy of each subtype, the percentages of IL-4Rα+ and IFN-γ+ cells were quantified in flow cytometry and the ratio of IL-4Rα/IFN-γ was calculated accordingly. Th2 T-cells were found to be the most predominant T-helper T-cells subtype in the collagen type I and MD hydrogels at all time points. In contrast, the native non-decellularized hydrogels were mostly Th1 dominant at all time points (FIG. 18C). These data also confirm the findings in the macrophage ratio analysis (FIG. 17B and FIG. 17C).

Collectively, these results indicate that the xenogeneic MD hydrogels stimulated a strong pro-remodeling immune response in the xenotransplantation animal model, suggesting their biocompatibility and the efficacy of the MD decellularization protocol described herein in reducing their overall immunogenicity.

The examples described herein demonstrated that, using porcine skeletal muscles as a tissue model, the decellularization protocol influences the characteristics of ECM-derived hydrogels. The MD decellularization protocol described herein significantly reduced the total DNA content without altering the biochemical composition of the ECM, which resulted in highly cyto-compatible and myo-inductive smECM hydrogels with substantially improved overall properties suitable for skeletal muscle regenerative engineering as compared to standard methods. The examples also demonstrated that decellularization alone appears insufficient to cleave xeno-antigens from animal ECM-derived materials, such as the α-Gal epitope, and that an post-decellularization treatment regimen may be important to efficiently cleave the xeno-antigens and reduce their overall immunogenicity. Comprehensive in vivo evaluations showed that smECM hydrogels decellularized by the MD protocol described herein are biocompatible and exhibit low immunogenicity as evidenced by their ability to modulate the immune response both by driving the macrophage response toward an M2 phenotype and by exhibiting anti-inflammatory and immunomodulatory effects, thereby inhibiting their rejection in a xenotransplantation model.

That such advantages over standard dECMs (and dECM methods) were possible was not previously known, nor would one of skill in the art have be led, based on the teaching in the art, to invent the methodology described and claimed herein.

### METHODS

### Method 1: Decellularization of Porcine Hindlimbs Skeletal Muscles

Skeletal muscle tissues were harvested from the hindlimb of skeletally mature porcine (Animals Technologies Inc.) under aseptic conditions and placed in sterile distilled water (DH₂O) containing 1% penicillin/streptomycin (P/S, Gibco, USA) on ice. In a sterile tissue culture hood, the muscle tissues were thoroughly purified by removing the fat tissues and blood vessels. Muscle tissues were then ground (two times) into small fragments using a manual meat grinder and placed in glass bottles containing sterile DH₂O and 1% P/S. The bottles were left under slow stirring at 4° C. for 1 hour to remove the remanent blood in the sample, followed by aspirating the washing solution in preparation for decellularization. Three different decellularization protocols were tested, one newly described herein and two known in the art: (a) freeze-thaw (mechanical disruption): muscle fragments were placed in a glass bottle containing sterile DH₂O and 1% P/S and subjected to 7 freeze-thaw cycles at −80° C. (12 hours freezing, followed by thawing at room temperature (RT) until completely thawed then stirring for 1 hour at RT). The DH₂O solution was replaced with a fresh one at each freeze-thaw cycle. (b) Triton X-100 (non-ionic detergent): muscle fragments were placed in a glass bottle containing 1% Triton X-100 (Bio-Rad, USA) solution in sterile DH₂O and 1% P/S and left under slow stirring for 3-days at RT with replacing the solution every 24 hours. (c) Sodium deoxycholate (ionic detergent): muscle fragments were placed in a glass bottle containing 0.2% Sodium deoxycholate (SDC, Sigma Aldrich, USA) solution in sterile DH₂O and 1% P/S and left under slow stirring for 3 days at RT with replacing the solution every 24 hours. Next, muscle fragments were separately placed in glass bottles containing sterile-filtered isopropanol (to eliminate residual lipids that could potentially interfere with gelation) and left under slow stirring for 24 hours at RT, followed by three washes with sterile DH₂O and 1% P/S. The muscle fragments were then incubated with 5×10⁷ U/l deoxyribonuclease I (DNase-I) and 1×10⁶ U/l ribonuclease (RNase) (all from Sigma Aldrich) solution in sterile DH₂O containing 1% P/S and left under slow stirring for 12 hours at RT, followed by three washes with sterile DH₂O and 1% P/S. After that, muscle fragments were incubated with 20 U/l α-galactosidase (Sigma Aldrich) solution in 1X phosphate buffered saline (PBS, Gibco) (pH 7.4) under slow stirring for 24 hours at RT to cleave the α-Gal epitopes in the tissue. Next, the solution was aspirated, and the muscle fragments were extensively washed in sterile DH₂O containing 1% P/S for 72 hours to remove any DNAse/RNAse and detergent residue, with replacing the solution every 24 hours. The muscle fragments were then frozen at −20° C. for 48 hours, followed by freeze-drying for 48 hours. Freeze-dried muscle fragments were then milled using a cryo-miller (6750, SPEX SamplePrep, USA) to obtain smECM in powder form. Native, non-decellularized skeletal muscles were freeze-dried and cryo-milled immediately after the grinding step to be used as controls. Finally, smECM powders were aliquoted and stored at −80° C. for later use.

Method 2: smECM Hydrogel Formation

smECM powders were digested at a stock concentration of 40 mg/mL in 30 mg/mL acetic acid (Bio-Rad) and 1 mg/mL pepsin (Sigma Aldrich) solution in DH₂O and incubated for 48 hours at RT under fast stirring. Next, the digested smECM solutions were neutralized to a pH of ˜7.4 using 1 N NaOH and salt-balanced using 10×PBS (all from Fisher Scientific, USA) on ice to prevent thermal gelation. To form smECM hydrogels, the neutralized smECM pre-gel solutions were diluted to a final concentration of 4 mg/mL using cold 0.5×PBS and incubated at 37° C. for 1 hour to induce gelation. Bovine corium collagen type I hydrogel (Collagen Solutions, UK), an extensively used hydrogel in skeletal muscle bioengineering, was prepared at a final concentration of 4 mg/mL using the same method described above and used as a control for some studies.

Method 3: smECM Hydrogel Quantitative Biochemical Characterizations

The biochemical composition (DNA, total protein, sulfated glycosaminoglycan (sGAG), and its collagen contents) within the finally-formed smECM hydrogels was quantified following biochemical quantification methods as described previously. First, 100 μL of each hydrogel was digested in 3 U/mL papain solution (Sigma Aldrich) for 16 hours at 60° C. before analysis (N=4/group/analysis). The measured values from each analysis were normalized to the dry weight of the smECM in the samples. DNA was isolated and quantified using a fluorometric double-strand DNA quantification kit (Quant-iT PicoGreen dsDNA reagent, Invitrogen, USA) following the manufacturer's instructions. Briefly, after papain digestion, samples were mixed with the PicoGreen reagent for 10-minutes in the dark at RT, followed by measuring the fluorescence signal using a spectrophotometer (Synergy H1, Bio-Tek) with an excitation of 485/20 nm and an emission of 528/20 nm. The total protein content was quantified using a BCA total protein kit (ThermoFisher, USA) following the manufacturer's instructions. Briefly, papain digested samples were mixed with the BCA reagent for 30 minutes at 37° C., followed by measuring the absorbance values at 562 nm as described above. The sGAG content was quantified by mixing the papain digested samples with 1,9-dimethyl-methylene blue (DMMB, Sigma Aldrich) and measuring the absorbance values at 525 nm as described above. The total collagen content was determined by measuring the hydroxyproline content in the papain digested samples using a hydroxyproline assay kit (Sigma Aldrich) after acid hydrolysis and reaction with p-dimethylaminobenzaldehyde and chloramine-T.

The growth factors content in the different smECM pre-gel solutions was analyzed using the Quantibody® Human Growth Factor Array Kit (RayBiotech, USA) following the manufacturer's instructions. smECM pre-gels at a final concentration of 1 mg/mL were used for the analysis. The kit's-stained array was scanned and analyzed by RayBiotech.

Method 4: smECM Hydrogel Qualitative Biochemical Characterizations

The biochemical composition (DNA, sGAG, collagen, α-Gal epitopes, laminin, and fibronectin contents) within the finally-formed smECM hydrogels were qualitatively visualized following histological and immunohistochemistry (IHC) assessments as described previously. First, smECM hydrogels were formed as described above in 5 mm3 molds (100 μL/mold), fixed with 10% buffered formalin (Fisher Scientific) and processed for paraffin embedding. Paraffin-embedded samples were sectioned into 6 μm thick sections and attached to glass slides.

For histological staining, slides were de-paraffinized, dehydrated, and stained as follows: hematoxylin & Eosin-Y and 4′,6-Diamidino-2-Phenylindole, Dihydrochloride (DAPI) were used to visualize the presence of any nuclei after decellularization. Briefly, slides were stained with hematoxylin (Sigma Aldrich) for 4 minutes, followed by a 10 minutes wash with running tap water. Next, slides were immersed in acid alcohol for 30 seconds and washed with tap water for 5 minutes. Finally, slides were stained with Eosin-Y (Sigma Aldrich) for 2 minutes. For DAPI (Invitrogen) staining, slides were incubated in a DAPI solution in PBS at a concentration of 1:3000 for 10 minutes in the dark. Alcian blue was used to assess sGAG content. Briefly, slides were stained with 1% Alcian Blue 8GX (Sigma Aldrich) in 0.1M HCL for 5 minutes, followed by three 30-seconds washes in DH₂O. Picrosirius red was used to assess collagen content and distribution. Briefly, slides were stained with sirius red (Sigma Aldrich) in a saturated aqueous solution of picric acid isopropyl alcohol for 1 hour and then incubated for 2 minutes (2×) in 0.5% acetic acid. After staining, all slides were dehydrated (expect for DAPI-stained slides) and cover-slipped using a mounting medium (ThermoFisher).

For IHC staining, slides were baked for 1 hour at 60° C. in an oven, de-paraffinized, rchydrated, and then processed for antigen retrieval through incubation in 10 mM sodium citrate (Sigma Aldrich) buffer at a 60°° C. water bath overnight. Next slides were blocked with 1% Bovine Serum Albumin (BSA, Sigma Aldrich) solution in 1× Tris Buffered Saline with tween (TBST, Bio-Rad) for 1 hour at RT prior to incubation with primary antibodies. Blocked slides were incubated with mouse anti-M86 (1:5 dilution, Enzo Life Sciences, USA), mouse anti-laminin and rabbit anti-fibronectin antibodies (1:500 and 1:400 dilutions, respectively, all from Novus Bio-Techne, USA) at 4° C. overnight. Slides were then washed in triplicate using 1× TBST and incubated with goat anti-mouse IgG (Alexa Fluor 594) and goat anti-rabbit IgG (Alexa Fluor 488) secondary antibodies (1:500 dilution, all from Abcam, USA) for 2 hours at RT. All primary and secondary antibodies were diluted using 1% BSA solution in 1× TBST. Finally, slides were washed in triplicate using 1× TBST and cover-slipped using Slow Fade diamond antifade mounting media (ThermoFisher). All slides were imaged using an inverted microscope (Leica DMi8, Leica, Germany). A polarized lens was used to visualize the collagen distribution in the picrosirius red-stained sections.

### Method 5: Gelation Kinetics

Gelation kinetics (gelation speed) was determined via turbidimetric spectrophotometric analysis as described previously. Briefly, 100 μL of neutralized pre-gel solutions from each group (N=4/group) was added into pre-cooled 96-well plates at 4° C. and then subsequently placed in a spectrophotometer pre-heated at 37°° C. The absorbance at 405 nm was measured every 1 minute for 60 minutes using the same plate reader described above. Absorbance values were normalized using the following equation: NA=(A−A₀)/(Aₘₐₓ−A₀), where NA is the normalized absorbance, A is the absorbance at any given time, A₀ is the initial absorbance, and Aₘₐₓ is the maximal absorbance. Normalized values were used to calculate the time required to reach 50% (t_{1/2}) and 95% (t₉₅) gelation, the lag phase (t_{lag}) (the time spent before the hydrogel starts gelling; calculated by extrapolating the x-intercept of the linear portion of the curve), and the gelation velocity(S) (determined by calculating the slope of the curve at t_{1/2}).

### Method 6: Scanning Electron Microscopy (SEM)

Hydrogels were formed as described above in 5 mm³ molds (100 μL/mold), fixed in 4% paraformaldehyde (PFA, Electron Microscopy Sciences, USA) solution at 4° C. for 24 hours, washed in triplicate with PBS, dehydrated, and then dried on a critical point drier (931. GL, Tousimis, USA). Next, samples were mounted on 15 mm stubs, gold sputter-coated using (Polaron E5100) for 3 minutes to eliminate surface charging and imaged using NanoSEM 450 (FEI Ltd, Japan) at a working distance of 5 mm and an acceleration voltage of 18 kV. Images were acquired at magnifications of 1000× and 3000×. The hydrogel's fibril density was determined using the ImageJ software (version 1.4 g, National Institute of Health, USA) by applying a fixed threshold value to all images, followed by calculating the mean intensity of the threshold regions within the images. Four-six different images from every sample (N=4/group) were used for the quantification.

### Method 7: Isolation and Culture of Primary SSCs

Primary satellite stem cells (SSCs) were isolated from the calf muscles of (3.0-3.5 kg, male) New Zealand white rabbits (Envigo, USA) in accordance with the guidelines and regulations approved by the University of Connecticut Health Center Institutional Animal Care and Use Committee (IACUC). Briefly, the calf muscles from both hindlimbs were harvested and rinsed in PBS containing 1% P/S to remove excess blood. The muscle tissues were then thoroughly purified by removing the facial tissues, epimysium, fat, and blood vessels. The muscle tissues were then longitudinally cut in half using a scalpel to expose the belly region of the muscle. Subsequently, the muscle fascicles were purified from the perimysium under the guidance of a dissecting microscope and collected. The muscle fascicles were then further purified by removing any visible intramuscular fat, blood vessels, or connective tissues, followed by mincing into 1 mm³. Next, the minced muscle tissue was digested in Dulbecco's Modified Eagle Medium-F: 12 (DMEM-F: 12, Gibco) containing 0.2% (w/v) collagenase type IV (Gibco) and 0.4% (w/v) dispase II (Sigma Aldrich, USA) for 2 hours at 37° C. under agitation. Next, an equal volume of DMEM-F: 12 containing 10% Fetal Bovine Serum (FBS, Gibco) and 1% P/S was added to the digested tissue to terminate the digestion reaction, followed by passing the digestion solution through a 100 μm cell strainer and centrifugation at 400 g for 5 minutes. The cells were then plated in 10 cm polyester dishes at a density of 600K cell/dish and maintained in a humidified tissue culture incubator at 37° C. and 5% CO₂ in DMEM-F: 12 growth medium (GM) containing 18% FBS, 10 ng/ml human recombinant Epidermal Growth Factor (rhEGF), 1 ng/ml human recombinant Basic Fibroblast Growth Factor (rhbFGF; all from R&D systems), 10 μg/ml human insulin (Gibco), 0.4 μg/ml dexamethasone (Sigma Aldrich), and 1% P/S. The medium was changed on days 4 and 7. Once 80% confluency was reached, the cells were detached using 0.25% EDTA-trypsin (Gibco), and further expanded in T-150 flasks and maintained as described above. Cells at the 2nd passage were used for all experiments. Differentiation medium (DM) composed of DMEM with high glucose (DMEM-HG, Gibco) supplemented with 5% Horse Serum (HS, Gibco) and 1% P/S was used to promote the formation of multinucleated myofibers. GM and DM were changed every 2 days for all experiments.

### Method 8: Cell Migration Assay

The migration of SSCs was assessed using a Chemotaxis 96-well Cell Migration Assay Kit (Chemicon, USA). Briefly, cells to be assayed were starved for 14-18 hours prior to the migration assay by culturing them in GM containing no growth factors nor FBS and maintained in culture as described above. Before detaching the cells for the assay, 150 μL of the digested smECM solutions were added to each well of the kit's 96-well feeder layer tray. 150 μL of 10% FBS, and 1 mg/mL pepsin and 30 mg/mL acetic acid in DH₂O were added as positive and negative controls, respectively. In addition, collagen type I hydrogel was added as a third control for comparison. Next, the starved cells were resuspended in growth factors and FBS-free GM at a concentration of 0.4 M cell/mL, and 100 μL of the cell suspension was added to each well of the kit's membrane chamber. The plate was covered and maintained as described above for 24 hours. After 24 hours, the migrated cells toward the different chemoattractants were quantified following the manufacturer's instructions, and the cell migration was reported as a relative fluorescence unit (RFU).

### Method 9: Cyto-Compatibility Assessments

SSCs were mixed with collagen I (positive control) and the different smECM pre-gels at a density of 1 M cells/mL, and 100 μL of each pre-gel solution was cast into 96-well plates and allowed to gel as described above before the addition of 200 μL of GM to each well. Cell viability was assessed using LIVE/DEAD cytotoxicity assay kit (Invitrogen, USA) at days 3 and 7 of culture. Briefly, the hydrogels were transferred to new wells, rinsed twice with PBS, and then incubated with 100 μL of the kit's staining solution (100 μL PBS, 0.05 μL calcein AM, and 0.2 μL ethidium homodimer-1) for 15 minutes at RT. Afterward, the samples were washed twice with PBS and imaged in PBS using an inverted fluorescence microscope (Zeiss LSM 880, Germany). Cell growth was assessed and quantified (N=4/group) using the CellTiter 96® AQueous One Solution Cell Proliferation Assay (MTS, Promega Inc, USA) at days 3 and 7 of culture. Briefly, the samples were transferred to new wells, washed twice with PBS, and then incubated with 200 μL of the MTS solution (40 μL MTS solution, 160 μL GM) for 2 hours at 37° C. Next, 100 μL from the supernatant of each sample was transferred to 96-well plates, and the absorbance was read in duplicate at 490 nm using the same plate reader described above. To negate the interface of MTS reagent with the samples, acellular hydrogels from each group were incubated with the MTS reagent, and their absorbance values were deducted from the absorbance values measured for each corresponding hydrogel. In addition, the cell-mediated contraction of the different hydrogels was monitored, and images were taken daily over 7 days. The acquired images were used to calculate the change in hydrogel area using ImageJ software (N=4/group), and the results were plotted as % of the change in hydrogel area overtime.

### Method 10: Myo-Inductivity Assessments

SSCs were mixed with collagen I (control) and the different smECM pre-gels at a density of 15 M cells/mL, and 100 μL of each pre-gel solution was cast into 96-well plates and allowed to gel as described above before the addition of 200 μL of GM to each well. Cell differentiation was induced at day 4 of culture. Myofiber formation was then evaluated through Immunofluorescence staining. Briefly, samples were washed once with PBS, fixed in 4% PFA for 15 minutes, then permeabilized in 0.1% Triton X-100 for 15 minutes. Samples were then washed twice in PBS and blocked in 1% BSA for 1 hour. Blocked samples were incubated with mouse anti-MHC (1:4 dilution, DSHB, University of Iowa, USA) for two hours, washed twice in PBS, and incubated in goat anti-mouse IgG (Alexa Fluor 594) (1:500 dilution) secondary antibody mixed with DAPI for two hours. All primary and secondary antibodies were diluted using 1% BSA. The entire staining process was carried out at RT. Myofiber formation within the different hydrogels was visualized in PBS using an inverted fluorescence microscope (Zeiss LSM 880).

### Method 11: In Vivo Study Design and Subcutaneous Injections

A total of 54 Balb/c mice (male, 6-8 weeks old, Charles River Laboratory, USA) was used for the in vivo subcutaneous study. The animals were equally divided between 3 groups (18 animals/group) and 3 time points of days 3, 7, and 14 (6 animals/timepoint). In addition to testing the hydrogels prepared by the MD protocol, 2 additional groups were added for comparison as follows: 1) Native smECM hydrogels (non-decellularized smECM hydrogels), and 2) collagen I hydrogels (positive control).

For the subcutaneous injection procedure, each mouse was anesthetized under isoflurane at a maintained concentration of 2%. The dorsal region of each mouse was shaved and disinfected with 70% ethanol-alcohol and betadine. Each mouse received four 350 μL evenly spaced subcutaneous injections in the dorsal region (two injections on the right and two on the left dorsal region). Post-subcutaneous injections, the animals were allowed to recover from the anesthesia under a warm lamp.

### Method 12: Sample Harvesting and Preparation

At days 3, 7, and 14-post hydrogel injection, the animals were euthanized using CO₂. and the death was confirmed by cervical dislocation. For each animal, the hydrogels injected in the right dorsal region were collected along with the adjacent dermal tissue for histological and immunohistochemistry analyses; whereas, the hydrogels injected in the left dorsal region were collected without the dermal tissue for flow cytometry analysis. Samples collected for histological and immunohistochemistry analyses were immediately embedded in an optimal cutting temperature (OCT) embedding media and frozen by submergence in a 2-Methylbutane (Sigma Aldrich) bath placed in liquid nitrogen. OCT-embedded samples were sealed inside of 50 mL conical tubes and stored at −80° C. for later sectioning. Samples collected for flow cytometry analysis were placed in ice-cold Hank's Balanced Salt Solution with (HBSS, Gibco) solution containing 1% P/S at the time of collection and processed within 1 hour.

### Method 13: Histology and Immunohistochemistry Analyses

OCT-embedded samples were cryo-sectioned to obtain 7 μm thick and transverse sections of the hydrogel and the adjacent dermal tissue. Cryosections were then mounted on charged glass slides (Fisher Scientific) and used for histological and IHC staining. For histological staining, hematoxylin and Eosin-Y was used to visualize the density of the infiltrated cells and the presence of any foreign body giant immune cells. Briefly, mounted sections were fixed in 4% PFA for 15 minutes at RT, washed with DH₂O for 5 minutes, then stained with hematoxylin for 3 minutes. Next, slides were washed in DH₂O (6 dips), immersed in 95% ethanol (6 dips) and stained with Eosin-Y for 30 seconds. Finally, the slides were dehydrated by immersion in 95% ethanol (6 dips), 100% ethanol (2×, 6 dips), then xylene (2X, 6 dips) and cover-slipped as described above. For IHC staining, slides were fixed in 4% PFA for 10 minutes at −20° C., washed twice in 1× TBST, then permeabilized in 0.3 Triton X-100 for 10 minutes at RT. Next, the slides were washed twice in 1× TBST and blocked with 1% BSA solution in 1X TBST for 1 hour at RT prior to incubation with primary antibodies. Blocked slides were then incubated with rabbit anti-CD86, rabbit anti-CD163, rabbit anti-CD4, and rabbit anti-CD8 (1:250 dilution, All from ThermoFisher) at 4° C. overnight. Slides were then washed in triplicate using 1× TBST and incubated with goat anti-rabbit IgG (1:500 dilution, Alexa Fluor 488) secondary antibody mixed with DAPI for 2 hours at RT. All primary and secondary antibodies were diluted using 1% BSA solution in 1× TBST. Finally, slides were washed in triplicate using 1× TBST and cover-slipped as described above. All slides were imaged using an inverted microscope (Leica DMi8).

### Method 14: Flow Cytometry Analysis

Harvested samples from each animal were grouped together, minced into 1-2 mm³ then subsequently digested in HBSS solution containing 0.2% (w/v) collagenase type I (Gibco), 0.4% (w/v) dispasc II, and 1% P/S for 2 hours at 37° C. Next, equal volumes of 10% FBS in PBS were added to terminate the digestion reaction, and the resultant solution was poured through a 70 μm cell strainer and then centrifuged at 3.5K RPM for 10 minutes. Afterward, the solution was aspirated, and the cells were blocked in 10% FBS in PBS for 10 minutes at RT. During blocking, 10 μL from each sample was taken for cell counting. Subsequently, the cells were equally aliquoted into 2 mL Eppendorf tubes and stained with the primary antibodies rabbit anti-CD86 (1:50 dilution), rabbit anti-CD163 (1:50 dilution), rabbit anti-CD4 (1:50 dilution), rabbit anti-CD8 (1:100 dilution), rabbit anti-arginase (1:100 dilution), rabbit anti-iNOS (1:50 dilution), rabbit anti-IL-4Ra (1:25 dilution), and rabbit anti-IFN-γ (1:50 dilution; all from ThermoFisher) for 30 minutes at 4° C. Next, the cells were washed twice with 10% FBS in PBS and incubated with goat anti-rabbit IgG (1:2000 dilution, Alexa Fluor 488) secondary antibody for 30 minutes at 4° C. All primary and secondary antibodies were diluted using 10% FBS in PBS. Finally, the cells were fixed with 4% PFA for 5 minutes at RT, washed twice with 10% FBS in PBS, and then transferred in PBS into round bottom 96-well plates for scanning. Cells were either scanned immediately or stored at 4° C. and then scanned within 3 days using the BioRad ZE5 cell analyzer machine.

### Method 15: Statistics

All quantitative data were expressed as mean±standard deviation. All statistical analyses were performed using the statistical software Prism GraphPad version 8 (GraphPad, USA). Statistical analyses were performed using the one-way analysis of variance (one-way ANOVA) and two-way analysis of variance (two-way ANOVA) with Tukey's post hoc test. Statistical significance was evaluated at *p<0.05, **p<0.01, ***p<0.001, and ****p<0.0001.

All U.S. and PCT patent publications and U.S. patents mentioned herein are hereby incorporated by reference in their entirety as if each individual patent publication or patent was specifically and individually indicated to be incorporated by reference. In case of conflict, the present application, including any definitions herein, will control.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

Adequacy of any particular element for practice of the teachings herein is to be judged from the perspective of a designer, manufacturer, seller, user, system operator or other similarly interested party, and such limitations are to be perceived according to the standards of the interested party.

In the disclosure herein, any element expressed as a means for performing a specified function is intended to encompass any way of performing that function including. Applicants thus regard any means which can provide those functionalities as equivalent to those shown herein. No functional language used in claims appended herein is to be construed as invoking 35 U.S.C. § 112 (f) interpretations as “means-plus-function” language unless specifically expressed as such by use of the words “means for” or “steps for” within the respective claim.

What is claimed is:

1. A method for making a decellularized skeletal muscle extracellular matrix (smECM), comprising:
purifying skeletal muscle tissue to substantially remove non-skeletal muscle tissue comprising blood vessels, fat and facia;
grinding the skeletal muscle tissue into discrete fragments;
decellularizing the skeletal muscle tissue by mechanical disruption;
digesting nucleic acid present in the skeletal muscle tissue with a DNAse and a RNAse;
digesting galactose-alpha-1,3-galactose (α-Gal) antigen present in the skeletal muscle tissue with α-galactosidase;
lyophilizing the skeletal muscle tissue; and
cryo-milling the lyophilized skeletal muscle tissue to generate decellularized smECM in a powdered form.

2. The method of claim 1, wherein no detergent is used for decellularization of the skeletal muscle tissue.

3. The method of claim 1, wherein the skeletal muscle tissue is from a non-human mammal.

4. The method of claim 3, wherein the non-human mammal is a pig.

5. The method of claim 1, wherein the mechanical disruption comprises at least one freeze-thaw cycle.

6. The method of claim 1, further comprising:
digesting the decellularized smECM with a protease;
adjusting pH of the decellularized smECM to about 7.4 to make a low temperature, decellularized smECM pre-gel; and
incubating the low temperature, decellularized smECM pre-gel at about 37° C. to form a decellularized smECM hydrogel.

7. The method of claim 1, further comprising:
digesting the decellularized smECM with a protease; and
modifying the decellularized smECM with phenolic functional groups to make a phenolic functionalized, decellularized smECM (smECM-PhF).

8. The method of claim 6, further comprising:
combining horseradish peroxidase with smECM-PhF to make a smECM-PhF pre-gel; and
reacting the smECM-PhF pre-gel with hydrogen peroxide to form a smECM-PhF hydrogel.

9. A decellularized smECM produced according to the method of claim 1.

10. A decellularized smECM-PhF produced according to the method of claim 7.

11. A decellularized smECM hydrogel produced according to the method of claim 6.

12. A decellularized smECM-PhF hydrogel produced according to the method of claim 8.

13. The decellularized smECM hydrogel of claim 11, wherein total smECM protein content is statistically equivalent to an equivalent smECM that is not decellularized.

14. The decellularized smECM hydrogel of claim 11, wherein sulfated glucosaminoglycan (sGAG) concentration is statistically equivalent to an equivalent smECM that is not decellularized.

15. The decellularized smECM hydrogel of claim 11, wherein at least one of time to start gelling, time to 50% gelation, or time to 95% gelation is statistically equivalent to time to start gelling, time to 50% gelation, or time to 95% gelation, respectively, for an equivalent smECM that is not decellularized.

16. A composition comprising the decellularized smECM hydrogel of claim 11.

17. A composition comprising the decellularized smECM-PhF hydrogel of claim 12.

18. A method for treating volumetric muscle loss (VML), comprising
injecting in a subject with VML a therapeutically effective amount of the composition according to claim 16 at at least one site of VML in the subject.

19. A method for treating volumetric muscle loss (VML), comprising
implanting in a subject with VML a therapeutically effective amount of the composition according to claim 16 at at least one site of VML in the subject,
wherein the implanted composition is pre-formed in a shape complementary to muscle absent due to VML.

20. A method for treating volumetric muscle loss (VML), comprising
injecting in a subject with VML a therapeutically effective amount of the composition according to claim 17 at at least one site of VML in the subject.
